SEQUENCE LISTING (4 sequences)

SEQ ID NO: 1 - DNA, 9 bp, Homo sapiens
  misc_feature (3)..(3): n is a, c, g, or t
  misc_feature (6)..(7): n is a, c, g, or t
gcnctnnag

SEQ ID NO: 2 - DNA, 55 bp, Homo sapiens
gatcaagcacctggagggctcttcagagcaaagacaaacactgaggtcgctgcca

SEQ ID NO: 3 - DNA, 20 bp, Homo sapiens
tggccacacgagtgccctca

SEQ ID NO: 4 - DNA, 120 bp, Homo sapiens
cccaccccgttctgttcccccacagtttagacaagatcctcatgctccactggccacacgagtgccctcaggaggagtagacacaggtggagggagctccttttgaccagcagagaaaac

(19) United States
(12) Patent Application Publication
(10) Pub. No.: US 2024/0318255 A1
(43) Pub. Date: Sep. 26, 2024

(54) TRANSCRIPTION FACTOR BINDING SITE ANALYSIS OF NUCLEOSOME DEPLETED CIRCULATING CELL FREE CHROMATIN FRAGMENTS

(71) Applicant: Belgian Volition SRL (BE/BE), Isnes (BE)

(72) Inventors: Jacob Vincent MICALLEF, London (GB); Mark Edward ECCLESTON, Cambridge (GB); Marielle HERZOG, Isnes (BE)

(21) Appl. No.: 18/260,011

(22) PCT Filed: Dec. 29, 2021
(86) PCT No.: PCT/EP2021/087814

§ 371 Date: Jun. 29, 2023

Related U.S. Application Data
(60) Provisional application No. 63/131,728, filed on Dec. 29, 2020.

(51) Int. Cl. C12Q 1/6886; G01N 33/53; G01N 33/68

(52) U.S. Cl. CPC C12Q 1/6886; G01N 33/5308; G01N 33/6854; G01N 33/6875

(57) ABSTRACT

The invention relates to methods for detecting disease in a subject by means of a minimally invasive body fluid test for non-nucleosomal cell free DNA fragments. The invention also relates to the measurement or detection of circulating cell free DNA fragments that include a transcription factor binding site sequence as an indicator of the presence of disease in a subject.

### FIELD OF THE INVENTION

The invention relates to a method for detecting disease in a subject by means of a minimally invasive blood test for transcription factor occupancy of cell free DNA fragments.

### BACKGROUND OF THE INVENTION

Cancer is a common disease with a high mortality. The biology of the disease is understood to involve a progression from a pre-cancerous state leading to stage I, II, III and eventually stage IV cancer. For the majority of cancer diseases, mortality varies greatly depending on whether the disease is detected at an early localized stage, when effective treatment options are available, or at a late stage when the disease may have spread within the organ affected or beyond when treatment is more difficult. Late stage cancer symptoms are varied including visible blood in the stool, blood in the urine, blood discharged with coughing, blood discharged from the vagina, unexplained weight loss, persistent unexplained lumps (e.g. in the breast), indigestion, difficulty in swallowing, changes to warts or moles as well as many other possible symptoms depending on the cancer type. However, most cancers diagnosed due to such symptoms will already be late stage and difficult to treat. Most cancers are symptomless at early stage or present with non-specific symptoms that do not help diagnosis. Cancer should ideally therefore be detected early using cancer tests.

To address the need for simple routine cancer blood tests, many blood borne proteins have been investigated as potential cancer biomarkers including carcinoembryonic antigen (CEA) for CRC, alpha-fetoprotein (AFP) for liver cancer, CA125 for ovarian cancer, CA19-9 for pancreatic cancer, CA 15-3 for breast cancer and PSA for prostate cancer. However, their clinical accuracy is too low for routine diagnostic use and they are considered to be better used for patient monitoring.

More recently, workers in the field have investigated circulating tumor DNA (ctDNA) as a blood based biomarker for cancer detection. Cell free DNA (cfDNA) circulates in the blood as chromatin fragments that are thought to originate from cell death, mainly by apoptosis, of a huge number of cells daily. During the process of apoptosis chromatin is fragmented into mononucleosomes and oligonucleosomes, some of which are released from the cells to circulate as cell free nucleosomes. Each circulating cell free nucleosome is associated with a small DNA fragment of less than 200 base pairs (bp) in length. Similarly, cell free chromatin fragments consisting of DNA bound transcription factors, or other non-histone chromatin proteins, in the circulation has been inferred from fragmentomics analysis. In healthy subjects circulating chromatin fragments are thought to be of hematopoietic origin and levels are low. Elevated levels of circulating nucleosomes, and hence cfDNA fragments, are found in subjects with a variety of conditions including many cancers, auto-immune diseases, inflammatory conditions, stroke and myocardial infarction (Holdenrieder & Stieber, 2009).

At least some of the cfDNA in the blood of cancer patients is thought to originate from the release of nucleosomes and other chromatin fragments into the circulation from dying or dead cancer cells (i.e. the cfDNA includes some ctDNA). Investigation of matched blood and tissue samples from cancer patients shows that cancer associated mutations, present in a patient's tumor (but not in his/her healthy cells) are also present in cfDNA in blood samples taken from the same patient (Newman et al, 2014). Similarly, DNA sequences that are differentially methylated (epigenetically altered by methylation of cytosine residues) in cancer cells can also be detected as methylated sequences in cfDNA in the circulation. In addition, the proportion of circulating cfDNA that is comprised of ctDNA is related to tumor burden so disease progression may be monitored both quantitatively by the proportion of ctDNA present and qualitatively by its genetic and/or epigenetic composition. Analysis of ctDNA can produce highly useful and clinically accurate data pertaining to DNA originating from all or many different clones within the tumor and which hence integrates the tumor clones spatially. Moreover, repeated blood sampling over time is a much more practical and economic option than, for example, repeated tissue biopsy. Analysis of ctDNA has the potential to revolutionize the detection and monitoring of tumors, as well as the detection of relapse and acquired drug resistance at an early stage for selection of treatments for tumors through the investigation of tumor DNA without invasive tissue biopsy procedures. Such ctDNA tests may be used to investigate all types of cancer associated DNA abnormalities (e.g.; point mutations, nucleotide modification status, translocations, gene copy number, micro-satellite abnormalities and DNA strand integrity) and would have applicability for routine cancer screening, regular and more frequent monitoring and regular checking of optimal treatment regimens (Zhou et al, 2017).

Blood plasma is commonly used as substrate for ctDNA assays. The cfDNA fragments (including any ctDNA) are extracted from the plasma (and hence removed from binding to nucleosomes, transcription factors or other proteins) and analyzed for nucleotide base sequence. Any DNA analysis method may be employed but typically analysis is performed by deep sequencing using Next Generation Sequencer instrumentation.

As DNA abnormalities are characteristic of all cancer diseases and ctDNA has been observed for all cancer diseases in which it has been investigated, ctDNA tests have applicability in all cancer diseases. Cancers investigated include, without limitation, cancer of the bladder, breast, colorectal, melanoma, ovary, prostate, lung liver, endometrial, ovarian, lymphoma, oral, leukaemias, head and neck, and osteosarcoma (Crowley et al, 2013; Zhou et al, 2017; Jung et al, 2010).

One example method of cfDNA analysis involves the identification of the tissue or cells of origin of the cfDNA fragments of a subject. The basis of this approach is that all cfDNA fragments present in the circulation have avoided digestion by nucleases during cell death or in the circulation because they are protected from nuclease action by protein binding within nucleosomes. The approach involves the determination of the nucleosome fragmentation pattern of cfDNA in a blood sample taken from the subject and locating the genomic position of the cfDNA fragments in a reference genome. The pattern of fragmentation differs for different cell types and can be used to identify the cells of origin of the cfDNA of the subject.

This approach involves extraction of cfDNA (including any ctDNA) from a plasma sample and whole genome sequencing of the DNA to detect the nucleosome bound DNA pattern displayed by the cfDNA fragments. The endpoint sequences of the cfDNA fragments are located for their genomic position within a reference genome or genomes using bioinformatics by computer analysis. The genomic locations of the cfDNA endpoints within the reference genome provides a map of the nucleosome protected cfDNA coverage of the genome.

The proportional contributions of different cell types or tissues to the cfDNA in a subject may also be determined by comparison of the nucleosome fragmentation patterns of the subject to calibration samples containing known relative abundance of cfDNA from different cellular sources using bioinformatics by computer analysis as described in WO2017012592.

The cfDNA fragments associated with chromatin fragments containing nucleosomes are typically 120-200 bp in length. However, protein binding and protection of cfDNA is not limited to the histone binding of cfDNA in nucleosomes. Other cfDNA fragments, including active gene promoter sequences, are bound by transcription factors, cofactors or other non-histone chromatin proteins either in addition to a nucleosome or in the absence of any nucleosome. In the absence of a nucleosome, these proteins often bind and protect shorter cfDNA fragments in the range of 35-80 bp. However, these shorter cfDNA fragments are only observed experimentally if the DNA fragment library preparation method used is suitable for the isolation, amplification and sequencing of short DNA fragments of less than 100 base pairs in length (Snyder et al, 2016).

The protein binding involved may be of different types. For example, some cfDNA sequences, including some inactive DNA sequences, are histone bound in a nucleosome conformation. The cfDNA fragments associated with chromatin fragments containing nucleosomes are typically of approximately 120-200 bp in length. Other cfDNA fragments, including active gene promoter sequences, are bound by transcription factors, cofactors or other chromatin proteins and these proteins often bind and protect shorter cfDNA fragments in the range of 35-80 bp. However, these shorter cfDNA fragments are only observed experimentally if the DNA fragment library preparation method used is suitable for the isolation, amplification and sequencing of short fragments.

The pattern of protein binding of DNA across the genome in living cells varies with cell type because different DNA sequences, including different promoter sequences and gene sequences, are active in different cells. The pattern of protein binding of DNA in any cell type can be determined by Nuclease Accessible Site mapping by digestion of chromatin extracted from the cell with a nuclease enzyme and sequencing the undigested DNA in the resulting protein protected chromatin fragments. Thus, if one views the cfDNA fragments in the blood as the product of an in vivo nuclease digestion, the cfDNA sequences found should correspond to protein bound DNA sequences in the cell from which the cfDNA originated. In principle therefore, the pattern of cfDNA fragment sequences in the blood should be similar to the pattern of sequences of chromatin fragments generated by Nuclease Accessible Site mapping of the cells of origin. Thus, the fragmentation pattern of cfDNA sequences determined from a blood sample can be compared using bioinformatics methods to known DNA fragmentation patterns generated by Nuclease Accessible Site analysis of cells of known tissue or cancer type to determine the tissue of origin of the cfDNA. The results in samples taken from healthy subjects indicate that the cells of origin of cfDNA are hematopoietic. The results of this approach in samples taken from cancer patients indicate that the cfDNA and ctDNA originate from a mixture of cells including hematopoietic cells and other cells. In many cases the non-hematopoietic cell type indicated correlates with the tissue of the cancer disease of the patient (Snyder et al, 2016).

Other workers have used a similar cfDNA fragment endpoint analysis approach, but focused the bioinformatic computer analysis on transcription factor binding site (TFBS) sequences. The aim of this approach is to determine TFBS accessibility and identify TFBS DNA sequences with altered accessibility in plasma samples taken from patients with cancer (Ulz et al, 2019). In this approach, a blood plasma sample is taken from a subject and the cfDNA is extracted and amplified using a DNA library preparation method suitable for small DNA fragments of less than 100 bp in length. The DNA library is sequenced using a next generation sequencing method. The sequencing data is used to identify the cfDNA fragmentation pattern in the genomic region near to a TFBS using bioinformatics methods. The analysis involves determining the nucleosome positioning profile of cfDNA fragments across a TFBS and its flanking sequences in a gene promoter sequence to determine whether or not the TFBS was bound to a transcription factor in the chromatin fragments that comprised the cfDNA. The method is complex but can be summarized as follows:

If the cfDNA fragmentation pattern observed in the DNA sequences that span a TFBS and flanking sequences in the genome displays a periodicity of approximately 200 bp, this relates to alternating stronger protein binding protection (at the center of a nucleosome binding position) and weaker protein binding protection (between nucleosomes where the DNA is unbound and unprotected) of DNA from degradation. In this case, the TFBS and flanking sequences is assumed to have been nucleosome covered in the chromatin fragments that comprised the cfDNA in the plasma sample.

If the cfDNA fragmentation pattern present displays protein binding protection of a TFBS and its flanking sequences, but with no (or an attenuated) nucleosome related periodicity, this relates to transcription regulatory protein binding at the TFBS and its flanking sequences. In this case, the TFBS is assumed to have been bound to one or more transcription factors and/or other regulatory proteins in the chromatin fragments that comprised the cfDNA in the plasma sample.

In healthy subjects, the cfDNA fragmentation pattern found typically correlates with the pattern obtained for nuclease accessible site experiments of haemopoietic cells. Thus, the TFBS sequences that are transcription factor bound or nucleosome covered in the cfDNA correlate with transcription factors that are, or are not, expressed in haemopoietic cells. In cancer patients, the pattern relates to a mixture of cell types in which the TFBS may be transcription factor bound in the cancer cell type and nucleosome bound in the haemopoietic cell type. However, fragmentomics bioinformatics methods have been developed to disentangle the small transcription factor protected TFBS fragment signal present in ctDNA from the much greater superimposed nucleosome periodicity signal present in the hematopoietic derived cfDNA component. Fragmentomics analysis indicates that the mixed pattern includes cfDNA TFBS sequences that are transcription factor bound for transcription factors that are not expressed in haemopoietic cells, but expressed by the cancer tissue.

We have previously described immunoassay tests for circulating cell free nucleosomes containing particular epigenetic signals including particular post-translational modifications, histone isoforms, modified nucleotides and non-histone chromatin proteins for the detection of cancer and other diseases (as referenced in WO2005019826, WO2013030577, WO2013030579 and WO2013084002). We have also described immunoassay tests for chromatin fragments including transcription factor bound DNA for the detection of cancer (as referenced in WO2017162755).

We now report improved methods for the analysis of circulating cell free TFBS DNA sequences in cfDNA from which the background periodic nucleosome signal is removed. These methods are suitable for use in body fluid samples as non-invasive, or minimally invasive, tests for diseases including cancer, autoimmune diseases and inflammatory diseases.

### SUMMARY OF THE INVENTION

According to a first aspect of the invention, there is provided a method of detecting a cell free DNA chromatin fragment including all or a part of a transcription factor binding site sequence, optionally including flanking sequences, in a body fluid sample obtained from a human or animal subject which comprises the steps of:
(i) contacting the body fluid sample with a binding agent which binds to a nucleosome; and
(ii) analyzing the DNA from the body fluid sample not bound to the binding agent in step (i).

According to a second aspect of the invention, there is provided a method of detecting a cell free DNA chromatin fragmentation pattern in a body fluid sample obtained from a human or animal subject which comprises the steps of:
(i) contacting the body fluid sample with a binding agent which binds to a nucleosome; and
(ii) analyzing the DNA from the body fluid sample not bound to the binding agent in step (i).

According to a further aspect of the invention, there is provided a method of detecting a disease in a human or animal subject which comprises the steps of:
(i) contacting a body fluid sample obtained from the human or animal subject with a binding agent which binds to a nucleosome;
(ii) isolating the DNA not bound to the binding agent in step (i);
(iii) optionally amplifying the isolated DNA;
(iv) determining the sequence of the DNA; and
(v) using the presence of a transcription factor binding site DNA sequence, and
optionally flanking DNA sequences, in the DNA as a biomarker for determining the presence and/or the nature of a disease in the subject.

According to a further aspect of the invention, there is provided a method of detecting a disease in a human or animal subject which comprises the steps of:
(i) contacting a body fluid sample obtained from the human or animal subject with a binding agent which binds to a nucleosome;
(ii) isolating the DNA not bound to the binding agent in step (i);
(iii) optionally amplifying the isolated DNA;
(iv) detecting the DNA; and
(v) using the DNA level and/or DNA sequence and/or DNA fragmentation pattern detected in step (iv) as an indicator of the presence and/or the nature of a disease in the subject.

According to a further aspect of the invention, there is provided a method of detecting a disease in a human or animal subject which comprises the steps of:
(i) contacting a body fluid sample obtained from the human or animal subject with a binding agent which binds to a nucleosome;
(ii) isolating the DNA not bound to the binding agent in step (i);
(iii) detecting the isolated DNA by a hybridization method; and
(iv) using the presence or amount of DNA hybridized as an indicator of the presence and/or the nature of a disease in the subject.

According to a further aspect of the invention, there is provided a method for detecting or diagnosing a disease in an animal or a human subject which comprises the steps of:
(i) removing a nucleosome from a body fluid sample obtained from the subject;
(ii) detecting, analyzing or measuring DNA associated with a cell free chromatin fragment in the remaining sample; and
(iii) using the DNA level and/or DNA sequence and/or DNA fragmentation pattern detected in step (ii) to identify the disease status of the subject.

According to a further aspect of the invention, there is provided a method for assessment of an animal or a human subject for suitability for a medical treatment which comprises the steps of:
(i) removing a nucleosome from a body fluid sample obtained from the subject;
(ii) detecting, analyzing or measuring DNA associated with a cell free chromatin fragment in the remaining sample; and
(iii) using the DNA level and/or DNA sequence and/or DNA fragmentation pattern detected in step (ii) as a parameter for selection of a suitable treatment for the subject.

According to a further aspect of the invention, there is provided a method for monitoring a treatment of an animal or a human subject which comprises the steps of:
(i) removing a nucleosome from a body fluid sample obtained from the subject;
(ii) detecting, analyzing or measuring DNA associated with a cell free chromatin fragment in the remaining sample;
(iii) repeating the detection, analysis or measurement of DNA associated with a cell free chromatin fragment in the remaining sample after removal of a nucleosome from a body fluid sample obtained from the subject on one or more occasions; and
(iv) using any changes in the DNA level and/or DNA sequence and/or DNA fragmentation pattern detected in step (iii) compared to step (ii) as a parameter for any changes in the condition of the subject.

According to a further aspect of the invention, there is provided a kit for the detection of a cfDNA fragment sequence comprising a nucleosome binder and reagents for the amplification, sequencing and/or fragmentation pattern of DNA associated with said cfDNA sequence, optionally together with instructions for use of the kit in the method as described herein.

According to a further aspect of the invention, there is provided a method of treating a disease in a subject in need thereof, wherein said method comprises the following steps:
(i) contacting a body fluid sample obtained from the human or animal subject with a binding agent which binds to a nucleosome;
(ii) detecting or measuring a DNA fragment not bound to the binding agent in step (i);
(iii) using the presence, amount, sequence and/or fragmentation pattern of the DNA fragment as an indicator of the presence of the disease in the subject; and
(iv) administering a treatment if the subject is determined to have the disease in step (iii).

According to a further aspect of the invention, there is provided a method of detecting a disease state in a fetus in a body fluid sample obtained from a pregnant human or animal subject which comprises the steps of:
(i) contacting the maternal body fluid sample with a binding agent which binds to a nucleosome;
(ii) analyzing the DNA not bound to the binding agent in step (i); and
(iii) using the presence, amount, sequence and/or fragmentation pattern of the DNA as an indicator of the disease state of the fetus of the subject.

### BRIEF DESCRIPTION OF THE FIGURES

FIG. 1: A cartoon illustration of the co-binding of various transcription factors at the promoter sites of the surfactant protein B, thyroglobulin, thyroperoxidase and thyrotropin receptor (TSH receptor) genes. CRE: cyclic adenosine monophosphate response element; GABP: GA-binding protein; HNF-3: Hepatocyte nuclear factor 3; NF-1: Nuclear factor 1; PAX-8: Paired box gene 8; Runx2: Runt-related transcription factor 2; TRα/RXR dimer: Thyroid hormone receptor a/Retinoid X receptor dimer; TTF-1: Thyroid transcription factor 1 (also known as NK2 homeobox 1, NKX2-1); TTF-2: Thyroid transcription factor 2.

FIG. 2: A cartoon of an example of the DNA loop structure of a transcription complex, to illustrate co-binding of some of the various regulatory proteins involved in a transcription complex including, without limitation, general transcription factors (GTF), gene specific transcription factors (TF), co-factors, activators, repressors, mediators, DNA bending proteins and RNA Polymerase. The regulatory proteins are bound to regulatory DNA sequences located near to the gene as well regulatory sequences far from the gene, including promoter sequences, TATA box sequences, enhancer sequences and repressor sequences. Other regulatory proteins (for example chromatin remodeling proteins) as well as other regulatory sequences are possible.

FIG. 3: Western blot analysis of recombinant mononucleosomes adsorbed onto magnetic beads coated with an antibody directed to bind to histone H3. The results demonstrate dose dependent adsorption of mononucleosomes by methods of the invention.

FIG. 4: Nucleosome ELISA results for human plasma samples and solutions of recombinant mononucleosomes following immunoprecipitation of nucleosomes using uncoated magnetic beads or magnetic beads coated with an antibody directed to bind to histone H3. The results demonstrate that both naturally occurring human circulating nucleosomes and recombinant nucleosomes in solution were unaffected by uncoated magnetic beads but were quantitatively removed by immunoprecipitation using magnetic beads coated with an antibody directed to bind to histone H3.

FIG. 5: Normalised coverage of 9780 published CTCF TFBS loci by short cfDNA fragments (35-80 bp) or larger cfDNA fragments (135-155 bp or 156-180 bp). (a) Coverage of CTCF TFBS loci by a cfDNA sequence library obtained for a plasma sample collected from a CRC patient with nucleosome depletion by a method of the invention. (b) Coverage of the same sample without nucleosome depletion.

FIG. 6: Normalised coverage of 1041 published CTCF TFBS loci occupied by CTCF in cancer cells but not in normal cells, by short cfDNA fragments (35-80 bp) or larger cfDNA fragments (135-155 bp or 156-180 bp). (a) Coverage of cancer associated CTCF TFBS loci by a cfDNA sequence library obtained for a plasma sample collected from a CRC patient with nucleosome depletion by a method of the invention. (b) Coverage of the same sample without nucleosome depletion.

### DETAILED DESCRIPTION OF THE INVENTION

Transcription factors are involved in cancer and account for about 20% of all known oncogenes (Lambert et al, 2018). We have previously described the use of a chromatin fragment containing a tissue specific transcription factor as a biomarker in serum for the detection or diagnosis of a cancer in a subject. The tissue specificity of the transcription factor can be used to indicate the tissue of origin of a cancer. For example, the transcription factor TTF-1 is reported to be expressed in thyroid and lung tissue and not in other tissues. The presence of circulating chromatin fragments containing TTF-1 therefore indicates the tissue of origin is lung or thyroid. We also described immunoassay methods for the measurement of circulating cell free chromatin fragments containing transcription factors. This immunoassay involves a double-antibody (or other binder) method where one binder is directed to bind to a transcription factor and the other to bind to DNA associated with the transcription factor or to a nucleosome component included in a chromatin fragment. In one embodiment described, the binder targeted to bind to a transcription factor is immobilized on a solid phase to isolate the chromatin fragment containing the transcription factor (i.e. to immunoprecipitate the chromatin fragment). The isolated chromatin fragment is then detected using a second binder directed to bind to DNA. This immunoassay method is simple, low cost and non-invasive. We now report the use of an improved cfDNA analysis method for the detection of disease. The principle underlying the method involves the removal of chromatin fragments containing nucleosomes from a body fluid sample prior to analysing cfDNA fragments associated with the remaining chromatin fragments. By this means, the nucleosome component of the cfDNA fragmentation pattern is removed from a sample, leaving the small cfDNA fragments that do not include a nucleosome. The presence of a TFBS sequence present in the cfDNA after removal of nucleosomes indicates that the sequence was protected by binding to the transcription factor in question and/or other regulatory protein (and was not nucleosome bound). This method for TFBS profile analysis obviates the need to identify the cfDNA fragment endpoints and/or their genomic location and/or complex bioinformatic methods for the disentanglement of mixed nucleosome and transcription factor bound fragmentomics signals and facilitates methods of ctDNA testing not previously possible.

The total cfDNA fragmentation pattern of a sample is formed by all chromatin fragments present in a sample including both those that do, or do not, contain a nucleosome. The chromatin fragments of primary interest in the present invention are those that contain no nucleosome. Thus, it is the non-nucleosomal cfDNA fragments that are of primary interest in the present invention.

The principle underlying the invention, involves the detection of a cfDNA regulatory sequence that is bound to a regulatory protein in a sample, for example a TFBS sequence that is bound to a transcription factor, after removal of nucleosomes. The TFBS may bind to a transcription factor that is expressed at an elevated level in the cells of a diseased tissue, but is not bound to a transcription factor in hematopoietic tissues where it is nucleosome bound. A chromatin fragment that contains such a TFBS sequence that is bound by a transcription factor is therefore likely to be derived from a cell in the diseased tissue where it was associated with an active gene. On the other hand, the same TFBS sequence will be nucleosome bound in chromatin fragments of hematopoietic origin (in which tissue the gene is inactive). Thus, removing nucleosome bound cfDNA fragments from the sample, leaves transcription factor occupied TFBS cfDNA fragments in place. The presence or amount of the TFBS sequence (optionally with flanking sequences) in the remaining cfDNA is sufficient to establish that the TFBS was transcription factor bound in the sample, without any need for the identification of fragment endpoint sequences or their genomic location or for complex determination and interpretation of nucleosome binding strength periodicity. Moreover, removal of a large portion of total chromatin fragments means that TFBS sequences (optionally with flanking sequences) can be more easily detected in the remaining cfDNA due to a low background.

The method removes nucleosomes of healthy hematopoietic cell origin in all locations genome wide prior to DNA analysis and hence also removes their nucleosome generated periodic cfDNA fragmentation patterns. The remaining cfDNA fragments, after removal of nucleosomes, will include sequences that are non-histone protein bound in diseased cells, for example TFBS sequences bound by one or more transcription factors. This is useful because there are many transcription factors expressed in cancer cells and other diseased cells that are not expressed in hematopoietic cells and the presence of their binding sequences in cfDNA after removal of nucleosomes is indicative of the tissue or cell of cfDNA origin of disease. For example, if a transcription factor, and corresponding transcription factor binding site(s), is selected that is expressed in cancer cells but is not expressed in hematopoietic cells, then any cfDNA fragments detected in a patient sample that include all or part of the TFBS sequence and, optionally flanking sequences, are indicative of the presence of the cancer disease in the patient (because chromatin fragments derived from healthy hematopoietic cells containing all or parts of the same TFBS and flanking sequences are nucleosome covered and have been removed).

The method has the advantages of (i) greater analytical sensitivity for the detection of transcription factor bound cfDNA fragments, (ii) greater analytical sensitivity to disease derived cfDNA fragmentation patterns, (iii) obviating complex bioinformatics analysis of mixed signals derived from cfDNA of mixed cellular origins, (iv) removing a large part of the sequencing requirement (of the removed nucleosomes) which makes the method more amenable for routine clinical use for example by use of PCR primers to amplify known TFBS sequences rather than by next generation whole genome sequencing, (v) reducing the sequencing cost and importantly (vi) increasing the clinical accuracy and utility of the method.

The methods of the invention involve the separation or removal of nucleosome bound cfDNA fragments, prior to identification of TFBS sequences in the remaining cfDNA. This is achieved by immunoprecipitation of all or most of those nucleosomes in a body fluid sample prior to extraction and/or amplifying and/or sequencing of cfDNA.

Immunoprecipitation may be achieved using any nucleosome binder including anti-nucleosome antibodies or other nucleosome binders, such as those described in WO2021038010.

We have developed immunoprecipitation methods to remove all or most nucleosomes (of healthy and diseased cellular origin) from a body fluid sample prior to extraction and/or amplification and/or sequencing of remaining cfDNA in a sample. Immunoprecipitation may be achieved by use of an anti-nucleosome antibody which binds to nucleosomes per se, or all nucleosomes, or most nucleosomes. We have developed methods for this separation involving anti-nucleosome antibodies linked to magnetic beads and have shown quantitative removal of nucleosomes from blood plasma samples.

Therefore, according to a first aspect of the invention, there is provided a method of detecting a cell free DNA fragment including all or a part of a transcription factor binding site (TFBS) (or other non-histone protein binding site) sequence, optionally including flanking sequences, in a body fluid sample obtained from a human or animal subject which comprises the steps of:
(i) contacting the body fluid sample with a binding agent which binds to a nucleosome; and
(ii) analyzing the DNA in the body fluid sample not bound to the binding agent in step (i).

In a second aspect of the invention, body fluid samples taken from a subject may be analysed for cfDNA fragmentation patterns, for example to detect disease and to identify the cells or tissue affected. Prior removal of nucleosomes from the sample facilitates the analysis of the cfDNA fragmentation patterns around active transcription factor binding sites by removing interference from nucleosome fragmentation patterns. Therefore, according to a second aspect of the invention, there is provided a method of detecting a cell free DNA chromatin fragmentation pattern in a body fluid sample obtained from a human or animal subject which comprises the steps of:
(i) contacting the body fluid sample with a binding agent which binds to a nucleosome; and
(ii) analyzing the DNA not bound to the binding agent in step (i) to detect the chromatin fragmentation pattern.

In one embodiment, the chromatin fragmentation pattern detected may be compared, e.g. using bioinformatics methods, to known DNA/chromatin fragmentation patterns (i.e. reference fragmentation patterns). The known reference fragmentation pattern may have been generated by Nuclease Accessible Site analysis of cells of a known tissue or cancer type. The comparison can be used to determine the tissue of origin of the cfDNA.

In a further embodiment, the chromatin fragmentation pattern detected may be compared, e.g. using bioinformatics methods, to known DNA/chromatin fragmentation patterns generated previously by investigation of patients with a known disease state, for example healthy patients or patients with a known cancer disease. The comparison can be used to determine the disease status of the subject.

Therefore, in another aspect of the invention there is provided a cfDNA fragment in a body fluid which is not bound to a nucleosome, with a TFBS sequence, optionally including flanking sequences, as a biomarker of disease.

In one embodiment there is provided a multiplicity of cfDNA fragments in a body fluid which are not bound to a nucleosome, which include a combination or pattern of TFBS sequences, optionally including flanking sequences, which together are used as a biomarker of disease.

It will be clear to those skilled in the art that removal of nucleosomes derived from healthy and/or hematopoietic cells or tissue may be sufficient for the purposes of the invention. It is known in the art that cell free nucleosomes derived from diseased or fetal cells or tissues, are associated with DNA fragments of approximately 147 bp in length. These nucleosomes include no linker DNA. In contrast, cell free nucleosomes derived from healthy and/or hematopoietic cells or tissues are associated with longer DNA fragment sizes of approximately 167 bp which do include linker DNA. Surprisingly, separation of cell free nucleosomes associated with longer DNA fragment sizes which include linker DNA can be achieved. We have previously demonstrated this through the use of nucleosome binders that bind to nucleosomes containing linker DNA (with associated cfDNA fragment sizes of approximately 167 bp), but do not bind to cell free nucleosomes that do not contain linker DNA (with associated cfDNA fragment sizes of approximately 147 bp). These binders can be used to immunoprecipitate nucleosomes of healthy cell origin containing cfDNA fragments of 167 bp, whilst leaving diseased or fetal derived nucleosomes associated with smaller DNA fragments of sizes of approximately 147 bp that do not comprise linker DNA in solution (as described in WO2021038010).

Therefore, in one embodiment, the binding agent binds to a nucleosome containing linker DNA.

In one embodiment, there is provided a method of detecting a cell free DNA fragment including all or a part of a TFBS (or other non-histone protein binding site) sequence optionally including flanking sequences, in a body fluid sample obtained from a human or animal subject which comprises the steps of:
(i) contacting the body fluid sample with a binding agent which binds to a nucleosome containing linker DNA; and
(ii) analyzing the DNA in the body fluid sample not bound to the binding agent in step (i).

In another embodiment, there is provided a method of detecting a cell free DNA fragmentation pattern, in a body fluid sample obtained from a human or animal subject which comprises the steps of:
(i) contacting the body fluid sample with a binding agent which binds to a nucleosome containing linker DNA; and
(ii) analyzing the DNA in the body fluid sample not bound to the binding agent in step (i).

In preferred embodiments the binding agent that binds to nucleosomes containing linker DNA is all or a part of a histone H1 moiety or a chromatin binding protein including, without limitation, Chromodomain Helicase DNA Binding Protein (CHD), DNA (cytosine-5)-methyltransferase (DNMT), High mobility group or high mobility group box proteins (HMG or HMGB), Poly [ADP-ribose] polymerase (PARP) and proteins containing Methyl-CpG-binding domains (MBD), e.g. MECP2. In one embodiment, the binding agent binds to histone H1 or a component thereof. In further preferred embodiments, the binding agent is attached to a solid support or precipitated so that the bound nucleosomes may be removed from the sample (i.e. the sample not bound to the binding agent is collected and the associated DNA is analyzed, as described herein).

As described above, the invention facilitates the identification of regulatory protein bound regulatory DNA sequences in a sample, based on the presence of the sequence in cfDNA following removal of nucleosomes. Therefore, according to one embodiment of the invention, there is provided a method of detecting a regulatory DNA sequence (optionally including flanking sequences) that is bound to a regulatory protein in cell free DNA in a body fluid sample obtained from a human or animal subject which comprises the steps of:
(i) contacting the body fluid sample with a binding agent which binds to a nucleosome; and
(ii) analyzing the DNA not bound to the binding agent in step (i) to detect the regulatory sequence (optionally including flanking sequences).

DNA analysis methods may involve DNA isolation and amplification. Therefore, in one embodiment there is provided a method of detecting a cell free DNA chromatin fragmentation pattern in a body fluid sample obtained from a human or animal subject which comprises the steps of:
(i) contacting the body fluid sample with a binding agent which binds to a nucleosome;
(ii) extracting the DNA from the body fluid sample not bound to the binding agent in step (i); and
(iii) analyzing the extracted DNA to detect the chromatin fragmentation pattern.

In one embodiment, the associated DNA analysis involves the identification of the presence of a cfDNA fragment including a transcription factor binding site (TFBS) sequence and/or flanking sequence. In further preferred embodiments, the binding agent is attached to a solid support or precipitated so that it, and its attached nucleosomes, may be removed from the sample.

The DNA sequences in nucleosome depleted cfDNA samples may be analyzed by any method known in the art. In preferred embodiments a cfDNA library produced by ligation of adapter oligonucleotides to the DNA fragments is amplified using a PCR method. Adapter oligonucleotides may include primer sequences to facilitate amplification of a library by PCR.

Therefore, in one embodiment of the invention, there is provided a method of detecting a cell free DNA fragment including all or a part of a TFBS (or other non-histone binding site) sequence optionally including flanking sequences, in a body fluid sample obtained from a human or animal subject which comprises the steps of;
(i) contacting the body fluid sample with a binding agent which binds to a nucleosome;
(ii) isolating the DNA fragments not bound to the binding agent in step (i);
(iii) attaching an adapter oligonucleotide to the DNA fragments isolated in step (ii);
(iv) amplifying the DNA fragments; and
(v) detecting all or a part of a TFBS (or other non-histone binding site) sequence, optionally including flanking sequences, in the amplified DNA.

In another embodiment of the invention, there is provided a method of detecting a cell free DNA fragmentation pattern in a body fluid sample obtained from a human or animal subject which comprises the steps of;
(i) contacting the body fluid sample with a binding agent which binds to a nucleosome;
(ii) isolating the DNA fragments not bound to the binding agent in step (i);
(iii) attaching an adapter oligonucleotide to the DNA fragments isolated in step (ii);
(iv) amplifying the DNA fragments;
(v) sequencing the DNA fragments; and
(vi) detecting a cell free DNA fragmentation pattern.

In other embodiments PCR primers are used for DNA amplification. Degenerate primers may be designed to amplify all DNA sequences isolated in step (ii), or specific primers may be designed using software known in the art to amplify specific DNA sequences associated with a TFBS of a transcription factor optionally also including flanking regions. The use of specific sequence primers means that the cfDNA can be analyzed for any particular TFBS sequence, optionally including flanking sequences, without any requirement for sequencing the whole cfDNA library.

Therefore, in one embodiment of the invention, there is provided a method of detecting a cell free DNA fragment including all or a part of a TFBS (or other non-histone binding site) sequence optionally including flanking sequences, in a body fluid sample obtained from a human or animal subject which comprises the steps of;
(i) contacting the body fluid sample with a binding agent which binds to a nucleosome;
(ii) isolating the DNA not bound to the binding agent in step (i);
(iii) amplifying the isolated DNA by a PCR method using sequence specific primers;
(iv) detecting the amplified DNA; and
(v) using the presence or amount of amplified DNA as an indicator of the presence of cfDNA fragments including all or a part of a TFBS (or other non-histone binding site) sequence optionally including flanking sequences in the sample.

A common method for identifying the DNA fragments of a selected sequence is by DNA hybridization to a complementary DNA sequence. Therefore, in another aspect of the invention, there is provided a method of detecting a cell free DNA fragment including all or a part of a TFBS (or other non-histone binding site) sequence optionally including flanking sequences, in a body fluid sample obtained from a human or animal subject which comprises the steps of;
(i) contacting a body fluid sample obtained from the human or animal subject with a binding agent which binds to a nucleosome;
(ii) isolating the DNA not bound to the binding agent in step (i);
(iii) optionally amplifying the DNA isolated in step (ii);
(iii) detecting the DNA by a hybridization method; and
(iv) using the presence or amount of DNA hybridization as an indicator of the presence of cfDNA fragments including all or a part of a TFBS (or other non-histone binding site) sequence optionally including flanking sequences in the sample.

The invention also provides a method of enriching or purifying transcription factor protected TFBS sequences in the cfDNA in a body fluid sample, by removing nucleosomal cfDNA prior to analysis of the cfDNA.

In one embodiment of the invention there is provided a method of detecting a transcription factor (or other non-histone protein) protected cfDNA sequence and/or flanking sequences in a body fluid sample obtained from a human or animal subject which comprises the steps of:
(i) contacting the body fluid sample with a binding agent which binds to a nucleosome; and
(ii) analyzing the cfDNA fragments not bound to the binding agent in step (i) for the presence of DNA sequences present in the TFBS (or other non-histone protein binding sequence) and/or flanking sequences

It will be understood that any non-histone protein which binds to DNA in chromatin may be suitable for use in methods of the invention, including transcription factors as well as other non-histone chromatin proteins including chromatin modifying proteins, genetic and epigenetic reading, writing and deleting proteins, proteins involved in RNA transcription (for example RNA polymerase molecules) and architectural or structural chromatin proteins (for example DNA bending proteins).

In one embodiment of the invention there is provided a method of detecting a DNA sequence protected by a non-histone protein in a body fluid sample obtained from a human or animal subject which comprises the steps of:
(i) contacting the body fluid sample with a binding agent which binds to a nucleosome; and
(ii) analyzing, measuring or sequencing the cfDNA fragments not bound to the binding agent in step (i)

In preferred embodiments, the binding agent is an antibody directed to bind to a nucleosome or a component thereof or a chromatin protein binder of nucleosomes. In preferred embodiments the binding agent is attached either directly or indirectly (for example by means of a linker system such as streptavidin/biotin) to a solid phase such as a plastic, magnetic plastic, sephadex, sepharose or other solid support known in the art. In other embodiments the binding agent is added as a liquid and isolated by cross-linking and precipitating the bound nucleosomes with polyethylene glycol (PEG) which can then be isolated as a solid phase precipitate, for example by centrifugation or filtration. Many immunoprecipitation methods are known in the art and any such methods may be useful in methods of the invention.

Methods of the invention have improved analytical sensitivity for transcription factor occupied TFBS sequences over previous methods described in the literature through reduced competing background signals for the detection of cfDNA fragmentation patterns at or near to TFBS sequences and flanking sequences. This is because disease derived cfDNA fragmentation patterns near to TFBS sequences may be poorly detected when obscured by nucleosome fragmentation patterns derived from healthy hematopoietic cells. Improvements in analytical sensitivity are important because some circulating cfDNA fragments including TFBS sequences may occur at low levels, near to, or below, the limits of detection by fragment endpoint analysis and other methods known in the art.

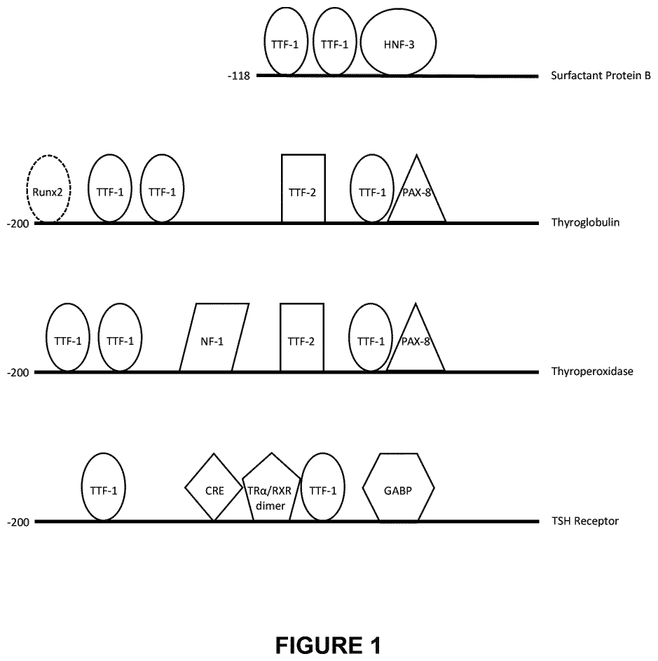

Methods of the invention also provide improved cfDNA tissue of origin specificity over previous methods described in the literature through improved methods for the detection of cfDNA transcription factor occupancy at or near to TFBS sequences and flanking sequences in two ways; (i) by facilitating simultaneous multiple TFBS analysis and (ii) because a single transcription factor may regulate different genes through binding to different DNA sequences in different gene promoters in the genome in different cells. Thus, the presence of a TFBS and its flanking sequences in cfDNA indicates the cell type of origin as exemplified for the binding of transcription factor TTF-1, in combination with different cofactors and other transcription factors, to different promoter sequences of different genes in different tissues as shown in FIG. 1.

Gene expression is regulated by specific binding of transcription factors to short TFBS DNA sequences, also referred to as response elements or binding motifs. The binding site is typically, but not necessarily, located in a gene promoter region near to the transcription start site of the regulated gene. Transcription factors bind to the DNA in a sequence specific manner through a DNA Binding Domain (DBD). Typically, a TFBS sequence is 5-15 bp long within the promoter of its target gene and a transcription factor protein can usually bind to a set of similar DNA sequences with varying degrees of binding affinity. The length of DNA fragments associated with circulating chromatin fragments containing transcription factors will vary depending on whether the fragment also includes further DNA protected sequences bound by further transcription factors, cofactors, nucleosomes or other chromatin proteins. Many such chromatin fragments are reported to contain cfDNA fragments in the 35-80 bp range (Snyder et al, 2016). Furthermore, we note that this size range is similar to the size range of chromatin fragments produced by nuclease digestion of chromatin extracted from the cells of cancer patients (Corces et al, 2018). We conclude that these cfDNA fragments of 35-80 bp are longer than typical DNA response elements and therefore include flanking DNA sequences. However, the DNA fragment size associated with a nucleosome typically exceeds 100 bp DNA. We therefore conclude that the cfDNA fragments shorter than 100 bp do not include intact nucleosomal DNA fragments. It is this pool of chromatin fragments consisting of transcription factors and other DNA binding chromatin proteins that do not comprise a nucleosome and which are associated with a cfDNA fragment in the 35-80 bp size range that is primarily addressed by the invention, in which all or most cell free nucleosomes are removed from a sample regardless of their linker DNA composition or tissue of origin.

It has been reported that a large part or most of the short cfDNA fragments of less than 100 bp in length do not derive from chromatin fragments including regulatory proteins, but derive from nucleosome associated DNA which is nicked or broken in one or both DNA strands. In this case the short cfDNA fragments may represent, for example, a 150 bp DNA fragment associated with a nucleosome which is nicked in one or more places to generate two or more smaller cfDNA fragments (for example two fragments of 75 bp) rather than a single 150 bp cfDNA fragment (Sanchez et al, 2018). Therefore, methods of the invention have the additional advantage of removal of short cfDNA fragments of less than 100 bp that originate from nucleosome associated nicked DNA. This further reduces the background of the nucleosome related cfDNA signal in the sample which enhances the sensitivity of the method for cfDNA fragments associated with transcription factor (or other non-histone protein) bound sequences.

The methods of the invention remove nucleosomal DNA with intact or nicked DNA and are therefore superior to current methods in the art for the separation of (isolated) DNA fragments on the basis of DNA size because, as well as being expensive and impractical for high throughput use, these methods fail to remove short cfDNA fragments of cell free nucleosomal nicked DNA origin.

Embodiments of the invention employing methods that remove all or most nucleosomes address cfDNA fragments of disease origin, regardless of whether or not the associated DNA fragment size is typical of a nucleosome associated DNA fragment.

Embodiments of the invention employing methods that remove nucleosomes containing linker DNA address predominantly cfDNA fragment sizes below 147 bp in length.

The response element of a transcription factor may occur repeatedly in many locations within the genome, and occurs in thousands of locations for some transcription factors. There is, therefore, the potential for the same transcription factor to be bound in a great many locations within the chromatin of a cell. This means that the death of a single cell may, in principle, give rise to a large number of circulating chromatin fragments containing the same transcription factor.

Moreover, transcription factors tend not to act alone but in concert with other transcription factors or co-factors or other moieties that are required for the regulation of a particular gene. Thus, a transcription factor may bind to a response element in the promoters of a large number of different genes, each in concert with different transcription factors. Thus, the DNA flanking sequence surrounding the same or similar TFBS sequence or response element, for the same transcription factor, varies in the promoters of different genes because it includes the binding motifs for different combinations of transcription factors. This applies to all or most transcription factors.

In addition, the binding sequence of the response element itself may be degenerate so that the transcription factor may bind to a variety of different motif sequences. For example, the transcription factor TTF-1 is expressed in a tissue specific manner in healthy lung and healthy thyroid tissue. In lung, two protein TTF-1 factors bind to the promoter region of the lung-specific Surfactant Protein B (SPB) gene. The DNA binding sequence, or binding motif, of TTF-1 in the promoter of SPB is GCNCTNNAG (SEQ ID NO: 1) (where A, C, G and T denote the DNA bases adenine, cytosine, guanine and thymine respectively and N denotes any of these bases). The wider consensus promoter DNA sequence surrounding the TTF-1 binding is (-118)GATCAAGCACCTGGAGGGCTCTTCAGAGCAAAGACAAACACTGAGGTCGCTGC CA(-64) (SEQ ID NO: 2), where (-64) denotes the distance in bp from the SPB transcription start site. In the SPB promoter in lung tissue, TTF-1 binds in concert with the transcription factor Hepatocyte Nuclear Factor 3 (HNF3) as shown in FIG. 1 (Matys et al, 2006 and Bohinski et al; 1994).

In the thyroid, TTF-1 regulates a number of genes including thyroglobulin, thyroid stimulating hormone receptor and thyroperoxidase. The consensus binding sequence for TTF-1 in the promoter region of thyroglobulin gene is different to than that in lung and is reported as TGGCCACACGAGTGCCCTCA (SEQ ID NO: 3). In the promoter of the thyroglobulin gene, TTF-1 binds cooperatively with TTF-2, PAX8 and Runx2 transcription factors and the wider sequence including 50 bp flanking sequences at the 5′ and 3′ ends is CCCACCCCGTTCTGTTCCCCCACAGTTTAGACAAGATCCTCATGCTCCACTGGCCACA CGAGTGCCCTCAGGAGGAGTAGACACAGGTGGAGGGAGCTCCTTTTGACCAGCAGA GAAAAC (SEQ ID NO: 4). Similarly, TTF-1 also binds to the promoter regions of the thyroid stimulating hormone receptor and thyroperoxidase genes in concert with different cooperating transcription factors in each case. Thus, not only does the sequence of DNA surrounding the TTF-1 binding site in the promoter sequence of genes regulated in thyroid or lung tissue differ, but the cofactors associated with TTF-1, and hence the surrounding DNA sequence, also differs for binding to different genes in the same tissue as shown in FIG. 1 (Matys et al, 2006 and Maenhaut et al, 2015). This demonstrates that the detection of a TFBS sequence, together with flanking DNA sequences, in the cfDNA of a subject by a method of the invention is sufficient to identify the origin of the chromatin fragment as lung or thyroid.

There are thought to be approximately 1000-3000 human transcription factors each of which binds specific locations in the genome resulting in dynamic transcriptional changes that drive a vast array of cellular processes. We have illustrated the principle of the invention with respect to TTF-1 as one example. However, any transcription factor may in principle be used in methods of the invention. Even, transcription factors that are ubiquitously expressed in many cell types and bind discreet DNA sequences, for example Hox protein transcription factors, bind cooperatively with cofactors to uniquely bind to different sequences to regulate different genes in different tissues (Merabet and Mann, 2016, Mann et al, 2009). This means that all or most transcription factors may be used for the methods of the invention. For example, the estrogen receptor-α (ERα) transcription factor binds to more than a thousand binding sites or estrogen response elements (ERE) in the human genome in concert with combinations of at least 60 other transcription factors at different genomic locations (Lin et al, 2007). Similarly, the androgen receptor (AR) binds the androgen response element (ARE) associated with thousands of genes in concert with other cooperating transcription factors at thousands of distinct different sequence loci. Thus, methods of the invention may identify the tissue of origin of a chromatin fragment containing ERα or AR through the sequence of associated DNA even though these transcription factors are expressed in multiple tissues. This is true of many transcription other transcription factors including CTCF.

Moreover, the DNA loci bound in cancer cells often differ from those bound in healthy cells, so the identification of a cfDNA fragment containing a TFBS sequence, optionally including flanking sequences, in the circulation by methods of the invention, enables both the identification of a subject with a cancer and the identification of the cancer type, for example as a prostate cancer or a lung cancer etc. (Pomerantz et al, 2015). This is enabled because chromatin is remodeled during tumorigenesis and this remodeling involves upregulation of tumor associated proteins through remodeled transcription factor binding patterns in the cancer cell. Because of this, the expression of many transcription factors is upregulated in cancer cells. This is a broad phenomenon, but can be exemplified by a few, non-limiting examples. For example, the well-known cancer associated transcription factors c-Myc and p53 are upregulated in most cancers. The binding site sequences bound by AR are greatly altered in prostate cancer (Pomerantz et al 2015). Similarly, the epithelial to mesenchymal transition (EMT) in cancer cells, which is associated with metastasis and resistance to therapy, involves the upregulation of the Jun/Fos family of transcription factors, including Fosll, Fosb, Fos, and Junb. The ETS (E26 transformation-specific) family of transcription factors as well as the Runxl, Tead and Nfkb transcription factors, have also been found to be highly enriched in the open chromatin of tumor cells. In addition, p63, Klf, Grhl, and Cepba are reported to be upregulated in tumor cells, and their binding sites are enriched in the open chromatin regions. Klf5 and p63 transcription factors are associated with carcinomas and act as drivers in lung and head and neck carcinomas. Further transcription factors associated with EMT include bHLH, Runx, Nfat, Tbx1, Tcf711 and Smad2 (Latil et al, 2017)

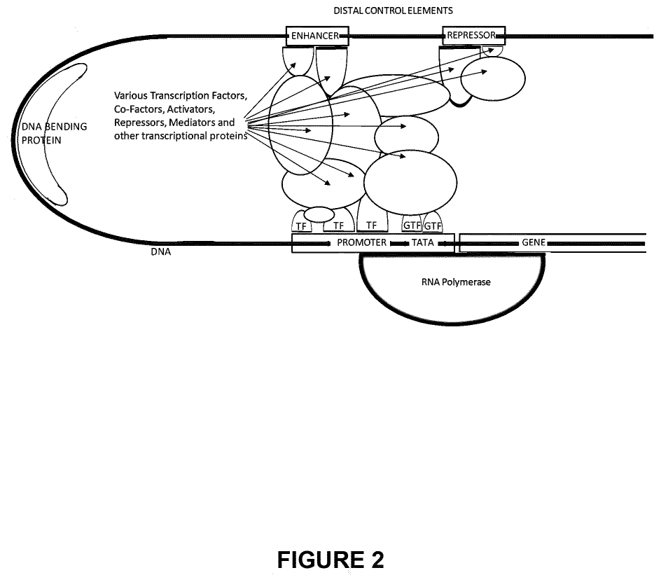

The regulation of transcription of eukaryotic genes involves a multiplicity of regulatory proteins bound to a multiplicity of regulatory DNA sequences, located both near to the transcription start site (TSS) of the gene and distal to the TSS in the genome in a transcription complex, for example as illustrated in FIG. 2. The distal regulatory sequences in the DNA may be located a few hundred to more than a million bases from the TSS or may be more distant. The transcription complex typically involves a loop of DNA, which may involve a DNA bending protein, wherein the more distal regulatory sequences, as well as the regulatory proteins bound to them, are brought into contact with the proteins that are bound to the regulatory sequences nearer to the TSS, for example as illustrated in FIG. 2. The TATA box is so named because it contains a sequence of repetitive Thymine/Adenine nucleotides that bind to general transcription factors required for transcription. Further gene specific transcription factors are also required for the expression of the particular gene (for example the transcription factors required to express the surfactant protein B, thyroglobulin, thyroperoxidase and TSH receptor genes as shown in FIG. 1). In addition, a multiplicity of other proteins are necessary including, for example without limitation, co-factors, mediators, activators, co-activators, repressors, co-repressors, chromatin remodeling proteins, DNA bending proteins, insulators and others. Such complexes may also include lengths of nucleosome protected DNA. Transcription complexes can be stable to facilitate high volume transcription. Therefore, circulating chromatin fragments of healthy and/or disease origin may include large protein/DNA complexes that comprise multiple proteins which may be resistant to nuclease activity. Some large transcription complexes involving near and distal regulatory sequences, as illustrated in FIG. 2, are termed super-enhancers. Super-enhancers are large clusters with high levels of transcription factor binding and are central to driving the expression of genes involved in controlling cell identity. Super-enhancers are also central to stimulating transcription of oncogenes in cancer. Cancer cells acquire super-enhancers and cancerous phenotypes rely on abnormal transcription driven by super-enhancers. Therefore, detection of the presence of chromatin fragments including all or parts of super-enhancer complexes and/or combinations of cfDNA fragment sequences that correspond to the near and/or distal regulatory sequences of super-enhancers by the methods described herein provides a method of identifying the cellular origin of chromatin fragments including cancer cells of origin.

The loop of DNA in such a chromatin fragment may in principle either be intact, or may be digested at one or more locations, resulting in either (i) two circulating chromatin fragments corresponding to the near and distal regulatory sequences; or (ii) a large chromatin fragment that contains two fragments of DNA. Therefore, cfDNA may include small DNA fragments that correspond to both the near and distal regulatory sequences of a gene.

In one embodiment, the disease is selected from cancer, an autoimmune disease or inflammatory disease. In a further embodiment, the disease is cancer. In a further embodiment, the autoimmune disease is selected from: Systemic Lupus Erythematosus (SLE) and rheumatoid arthritis. In a further embodiment, the inflammatory disease is selected from: Crohn's disease, colitis, endometriosis and Chronic Obstructive Pulmonary Disorder (COPD).

In preferred embodiments, the disease is cancer. In a further embodiment, the cancer is selected from: breast cancer, bladder cancer, colorectal cancer, skin cancer, melanoma, ovarian cancer, prostate cancer, lung cancer, pancreatic cancer, colorectal cancer, bowel cancer, liver cancer, endometrial cancer, lymphoma, oral cancer, pharynges, head and neck cancer, leukemia, lymphoma and osteosarcoma.

In another embodiment, the tissue affected by the disease is the organ of origin, such as the organ of origin of a cancer.

In another aspect of the invention, there is provided a method of detecting a disease in a human or animal subject which comprises the steps of:
(i) contacting a body fluid sample obtained from the human or animal subject with a binding agent which binds to a nucleosome;
(ii) isolating the DNA not bound to the binding agent in step (i);
(iii) amplifying the isolated DNA, for example by a PCR method;
(iv) determining the sequence of the amplified DNA; and
(v) using the presence of a transcription factor binding site DNA sequence, and optionally flanking DNA sequences, in the amplified DNA as a biomarker for determining the presence and/or the nature of a disease in the subject.

It will also be clear to those skilled in the art that a multiplicity of TFBS sequences with flanking sequences related to a multiplicity of gene promoter or other loci, may be obtained corresponding to various gene loci bound by one or more transcription factors and the data regarding the various sequences may be integrated to determine the nature of the disease and/or the tissue affected by the disease.

The DNA may be detected and analyzed using methods known in the art. Therefore, in one embodiment, the DNA is analyzed by PCR. For example, the DNA may be detected using a PCR method, such as a PCR method using adapters, degenerate primers or sequence specific primers. Alternatively, the DNA may be detected using a hybridization method, for example using a complementary sequence to capture the target sequence through hybridization.

In another aspect of the invention, there is provided a method of detecting a disease in a human or animal subject which comprises the steps of:
(i) contacting a body fluid sample obtained from the human or animal subject with a binding agent which binds to a nucleosome;
(ii) isolating the DNA not bound to the binding agent in step (i);
(iii) amplifying the isolated DNA, for example by a PCR method;
(iv) detecting the amplified DNA; and
(v) using the presence or amount of amplified DNA as an indicator of the presence and/or the nature of a disease in the subject.

The DNA sequences isolated in step (ii) may be amplified by any method known in the art. In preferred embodiments isolated DNA is amplified using a PCR method employing adapters which are ligated to the DNA fragments. In other embodiments PCR primers are used for DNA amplification. Degenerate primers may be designed to amplify all DNA sequences isolated in step (ii), or specific primers may be designed using software known in the art to amplify specific DNA sequences associated with the sequence of a response element of a transcription factor optionally also including flanking regions.

Therefore, in another aspect of the invention, there is provided a method of detecting a disease in a human or animal subject which comprises the steps of:
(i) contacting a body fluid sample obtained from the human or animal subject with a binding agent which binds to a nucleosome;
(ii) isolating the DNA not bound to the binding agent in step (i);
(iii) amplifying the isolated DNA by a PCR method using sequence specific primers;
(iv) detecting the amplified DNA; and
(v) using the presence or amount of amplified DNA as an indicator of the presence and/or the nature of a disease in the subject.

The presence or amount of DNA may be detected by a hybridization method. Therefore in one embodiment of the invention, there is provided a method of detecting a disease in a human or animal subject which comprises the steps of:
(i) contacting a body fluid sample obtained from the human or animal subject with a binding agent which binds to a nucleosome;
(ii) isolating the DNA not bound to the binding agent in step (i);
(iii) detecting the DNA by a hybridization method; and
(iv) using the presence or amount of DNA hybridized as an indicator of the presence and/or the nature of a disease in the subject.

In preferred embodiments the isolated DNA is amplified prior to hybridization. In preferred embodiments the hybridization is a multiplex method in which multiple DNA sequences are immobilized on a solid phase for the simultaneous binding of multiple TFBS sequences, optionally including flanking sequences. This allows for the testing of multiple TFBS sequences, and multiple disease conditions, in a single multiplex format. In preferred embodiments, the multiplex hybridization method is a DNA microarray or DNA chip method. Any multiplex method suitable for the investigation of multiple gene sequences may be used for methods of the invention. Many such methods are known in the art including the Luminex bead method (Dunbar, 2006).

A further method for detecting the presence of cfDNA fragments including TFBS sequences in a cfDNA sample involves contacting the cfDNA sample with the transcription factor protein itself. The transcription factor will then bind to any DNA sequence that contains one or more of its TFBS sequences. The transcription factor bound DNA may be detected by any method known in the art including, without limitation, the use of DNA binders (for example, an anti-DNA antibody or a DNA chelating agent) or by a PCR or hybridization method. In one embodiment, the DNA is detected or measured using a general DNA binder such as an anti-DNA antibody or a DNA chelating or intercalating agents, for example, ethidium bromide and cyanine dyes such as SYBR green and SYBR gold.

For example, the presence of the prostate specific NKX3.1 TFBS sequence in a DNA fragment library prepared from a subject sample, following removal of nucleosomes, indicates the subject is positive for prostate cancer. Therefore, the DNA fragment library may be contacted with solid phase immobilized transcription factor NKX3.1 to bind DNA fragments containing a NKX3.1 TFBS sequence. Binding of DNA from the library to NKX3.1 is indicative of prostate cancer.

Therefore, in another aspect of the invention, there is provided a method of detecting a disease in a human or animal subject which comprises the steps of:
(i) contacting a body fluid sample obtained from the human or animal subject with a binding agent which binds to a nucleosome;
(ii) isolating the DNA not bound to the binding agent in step (i);
(iii) optionally, amplifying the DNA isolated in step (ii);
(iv) contacting the DNA obtained in step (ii) or (iii) with a transcription factor protein; and
(v) using the presence, amount or sequence of DNA bound to the transcription factor as an indicator of the presence and/or the nature of a disease in the subject.

In one embodiment the DNA not bound to the nucleosome binding agent isolated in step (ii) is contacted with a multiplicity of (i.e. more than one) transcription factor proteins so that multiple sets of TFBS are captured and can be analysed in a multiplex test. This method enables the testing for multiple transcription factors and multiple diseases in a single patient sample. For example, testing for DNA fragments binding to multiple transcription factors, each specific for one or more cancer diseases, optionally in addition to transcription factors expressed in many cancers, enables a test for the detection of many different cancer diseases in addition to identifying the tissue of the cancer in a single blood test. Methods for multiplex testing are well known in the art, for example, without limitation, the multiplex beads system of Luminex Corporation can be used to conduct large numbers of separate assays in a single sample (Dunbar, 2006).

Therefore, in another aspect of the invention, there is provided a method of detecting a disease in a human or animal subject which comprises the steps of:
(i) contacting a body fluid sample obtained from the human or animal subject with a binding agent which binds to a nucleosome;
(ii) isolating the DNA not bound to the binding agent in step (i);
(iii) optionally, amplifying the DNA isolated in step (ii);
(iv) contacting the DNA obtained in step (ii) or (iii) with a plurality of transcription factors; and
(v) using the presence, amount or sequence of DNA bound to different transcription factors as an indicator of the presence, nature, location and/or the affected tissue of a disease in the subject.

In one embodiment the method described here is used to identify the tissue of origin of a tumour of unknown origin. This may be performed in a body fluid test as described above or may be performed on a chromatin fragment library produced by fragmentation of tumor tissue chromatin material obtained at biopsy or surgery. Methods for chromatin fragmentation are well known in the art including, without limitation, by digestion with nuclease enzymes and by sonication. In the particular case of testing tissue, the removal of nucleosomes prior to exposure to the transcription factor(s) may not be necessary (provided the sample is not contaminated with chromatin from healthy cells).

Therefore, in another aspect of the invention, there is provided a method of detecting a disease in a tissue sample obtained from a human or animal subject which comprises the steps of:
(i) isolating chromatin from a tissue biopsy sample;
(ii) fragmenting the chromatin isolated in step (i);
(iii) extracting the DNA from the chromatin fragments obtained in step (ii);
(iv) contacting the DNA isolated in step (iii) with one or a plurality of transcription factors; and
(v) using the presence, amount or sequence of DNA bound to the transcription factor(s) as an indicator of the presence, and/or tissue of origin of a disease in the subject.

Body fluid samples taken from a subject may be analysed for cfDNA fragmentation patterns to detect disease and to identify the cells or tissue affected. Removal of nucleosomes facilitates the analysis of the cfDNA fragmentation patterns around active transcription factor binding sites with interference from nucleosome fragmentation patterns removed. Therefore, according to a further aspect of the invention, there is provided a method of detecting the presence, and/or tissue of origin of a disease in a human or animal subject which comprises the steps of:
(i) contacting a body fluid sample obtained from the subject with a binding agent which binds to a nucleosome;
(ii) analyzing the DNA not bound to the binding agent in step (i) to detect the cfDNA fragmentation pattern; and
(iii) using all or a part of the cfDNA fragmentation pattern as an indicator of the presence, and/or tissue of origin of a disease in the subject.

In preferred embodiments, the disease is cancer. In a further embodiment, the nature of the disease is the tissue affected by the cancer.

It is well known in the art that cfDNA of fetal origin, for example containing Y-chromosome sequences originating from a (XY) male fetus, circulates in the blood of pregnant animal and human (XX) mothers. This cfDNA has similarly been reported to comprise both cfDNA fragments of the length expected of nucleosome protected DNA fragments (approximately 160 bp) as well as shorter cfDNA fragments in the range 50 bp upwards. Moreover, it has been reported that maternal cfDNA fragments of less than 140 bp in length are enriched for cfDNA of fetal origin (Hu et al, 2019). Thus, methods of the invention are applicable not only to disease states of the subject from whom the sample was taken, but also to maternal/fetal investigations including prenatal testing of fetal conditions in maternal blood samples.

Therefore, in one embodiment of the invention, there is provided a method of detecting a disease state in a fetus in a body fluid sample obtained from a pregnant human or animal subject which comprises the steps of:
(i) contacting the maternal body fluid sample with a binding agent which binds to a nucleosome;
(ii) analyzing the DNA not bound to the binding agent in step (i); and
(iii) using the presence, amount, sequence or fragmentation pattern of the DNA as an indicator of the disease state of the fetus of the subject.

### Nucleosome Binding Agents

Any moiety that binds to nucleosomes may be used for methods of the invention. In preferred embodiments of the invention in which all or most nucleosomes are removed prior to cfDNA analysis, the nucleosome binding agent is an antibody directed to bind specifically to a nucleosome. The antibody may be directed to bind to any nucleosome epitope or any component of a nucleosome. In preferred embodiments the antibody selected binds to a component present in all or most circulating cell free nucleosomes so that all or most nucleosomes are removed from body fluid samples prior to cfDNA analysis by the methods described herein.

In preferred embodiments the nucleosome binding agent is directed to bind to a nucleosome core epitope. The core histones H2A, H2B, H3 and H4 all feature core domains as well as histone tails of approximately 20-30 amino acids in length. The histone tails of circulating cell free nucleosomes may be wholly or partially removed to produce “clipped” histones. This is thought to be commonly caused by the action of endopeptidase cathepsin-L which is involved in the initiation of protein degradation. For example, cathepsin-L removes the histone H3 tail at amino acid position 21. Thus, an antibody directed to bind to histone H3 at an epitope located between amino acids 1-21 may fail to remove a nucleosome containing histone H3 in which the tails have been clipped. In our own experiments, we have observed that antibodies directed to bind histone H3 epitopes located at amino acid position 4-8 in the histone tail bind fewer nucleosomes than antibodies directed to bind epitopes located at amino acid positions above 21. Similar limitations will occur for the other core histones (i.e. H2A, H2B and H4). In our own method development we have used antibodies directed to bind to core histone H2A and H2B epitopes and histone H3 epitopes located at amino acids 30-33.

In one embodiment, the method additionally comprises using the presence, amount or sequence of the DNA as an indicator of the disease state of the subject. Therefore, in a preferred embodiment of the invention, there is provided a method of detecting a disease state in a body fluid sample obtained from a human or animal subject which comprises the steps of:
(i) contacting the body fluid sample with a binding agent which binds to a nucleosome core epitope;
(ii) analyzing the DNA not bound to the binding agent in step (i); and
(iii) using the presence, amount, sequence or fragmentation pattern of the DNA as an indicator of the disease state of the subject.

In embodiments of the invention in which nucleosomes containing linker DNA are removed prior to cfDNA analysis, the binding agent that binds to nucleosomes containing linker DNA is all or a part of a chromatin protein including a histone H1 moiety or a chromatin binding protein including, without limitation, Chromodomain Helicase DNA Binding Protein (CHD), DNA (cytosine-5)-methyltransferase (DNMT), High mobility group or high mobility group box proteins (HMG or HMGB), Poly [ADP-ribose] polymerase (PARP) and proteins containing Methyl-CpG-binding domains (MBD), e.g. MECP2. The binding agent may also be an antibody or other binder directed to bind to histone H1.

Binding agents used for methods of the invention may be coated on a solid support, such as sepharose, sephadex, plastic or magnetic beads. In one embodiment, said solid support comprises a porous material. In another embodiment the binding agent is derivatized to include a tag or linker which can be used to attach the binding agent to a suitable support which has been derivatized to bind to the tag. Many such tags and supports are known in the art (e.g. Sortag, Click Chemistry, biotin/streptavidin, his-tag/nickel or cobalt, GST-tag/GSH, antibody/epitope tags and many more). Isolation of the binding agent may then be performed prior to, concurrently with, or following the reaction of the binding agent with a nucleosome. For ease of use, the coated support may be included within a device, for example a microfluidic device.

In other embodiments the binding agent is added in solution and isolated by cross-linking and precipitating the bound nucleosomes with a precipitation agent such as polyethylene glycol (PEG). The precipitated pellet can then be isolated as a separate phase, for example by centrifugation or filtration. Many immunoprecipitation methods are known in the art and any such methods may be useful in methods of the invention.

### DNA Sequencing

There are many methods known in the art to analyze or identify a DNA sequence and any DNA analysis method may be employed for methods of the current invention including, without limitation, next generation sequencing methods, isothermal DNA amplification, cold PCR (co-amplification at lower denaturation temperature-PCR), MAP (MIDI-Activated Pyrophosphorolysis), PARE (personalized analysis of rearranged ends), DNA hybridization methods (including gene chip methods and in situ hybridization methods). In addition, the gene sequence may also be analyzed for epigenetically altered DNA sequences by epigenetic DNA sequencing analysis (e.g. for sequences containing 5-methylcytosine using bisulfite conversion of unmodified cytosine to uracil). Therefore, in one embodiment, cfDNA is analyzed using DNA sequencing, for example a sequencing method selected from Next Generation Sequencing (targeted or whole genome) and methylated DNA sequencing analysis, BEAMing, PCR including digital PCR and cold PCR (co-amplification at lower denaturation temperature-PCR), isothermal amplification, hybridization, MIDI-Activated Pyrophosphorolysis (MAP) or Personalized Analysis of Re-arranged Ends (PARE).

### DNA Library Preparation

The cfDNA present in a sample following removal of nucleosomes, may be amplified for ease of detection and sequencing using PCR methods. Methods for cfDNA fragment library preparation are well known in the art and typically involve the ligation of adapter oligonucleotides to the cfDNA fragments. The adapter oligonucleotide ligated DNA fragment library is then typically amplified by PCR. Degenerate PCR primer oligonucleotide sets may also be used to amplify cfDNA.

In principle, any library preparation method may be suitable for use with methods of the invention. Library preparation methods may involve amplification of single-stranded or double-stranded adapter ligated cfDNA fragments. Preferred library preparation methods involve single-stranded cfDNA adapter ligation. Preferred library preparation methods have high efficiency for amplification and isolation of small DNA fragments of less than 100 bp in length. Many such library preparation methods are known in the art including for example, (i) the TruSeq DNA Sample preparation Kit (Illumina) used according to the manufacturer's protocol with 20-25 PCR cycles for 5-10 ng of input DNA (Ulz et al; 2019), (ii) use of the MagMAX cfDNA Isolation Kit (Applied Biosystems) followed by library preparation using the NEBNext Ultra II DNA Library Prep Kit (New England Biolabs) (Ulz et al; 2019), (iii) use of the blood and body fluid protocol for the Qiagen QIAamp DSP DNA Blood Mini Kit with PCR amplification using the Life technologies Ion Plus Fragment Library Kit (Hu et al, 2019). Other methods include those described by Sanchez et al, 2018, Skene and Henikoff, 2017, Snyder et al, 2016 and Liu et al, 2019. In preferred embodiments the adapter oligonucleotides are ligated to the DNA fragments and are used to amplify all adapter ligated DNA fragments in a library. These methods are well known in the art.

PCR primers used for DNA amplification may also be of random sequence to amplify all sequences present in a library, or may be designed using software known in the art to amplify specific DNA sequences associated with the sequence of a response element of a transcription factor optionally also including flanking regions.

Alternatively, specific cfDNA sequences, for example associated with a response element of a transcription factor optionally also including flanking regions, may be amplified using specific primer oligonucleotides designed by methods known in the art. In this embodiment cfDNA fragments including TFBS sequences, optionally including flanking sequences, may be detected with no requirement for sequencing per se (for example Next Generation sequencing).

### Sample Preparation

The sample may be any body fluid in which chromatin fragments can be detected. Chromatin fragments are known to occur in blood, feces, urine and cerebrospinal fluid. We have also detected chromatin fragments in sputum. In preferred embodiments, the body fluid sample is a blood, serum or plasma sample. In highly preferred embodiments the sample is a blood plasma sample including a plasma sample collected in an EDTA blood collection tube or a plasma sample collected in a tube recommended for cfDNA analyses. Such tubes include, without limitation, cell-free DNA blood collection tubes produced by Roche, PAXgene, Norgene, LBgard and others. These samples may be used to measure and analyze circulating cfDNA fragments. For example, plasma samples such as EDTA plasma samples may be used in methods of the invention. The plasma may be used freshly or frozen until analyzed. In our own method development, we have used blood plasma samples collected in standard EDTA blood collection tubes and centrifuged within 2 hours. Our experimental results indicate that cell-free DNA blood collection tubes are also suitable.

Transcription Factors and their DNA Binding Sites

The regulation of gene transcription in eukaryotic organisms may be highly complex and involves bending and looping of DNA to bring together multiple regulatory DNA sequences bound by multiple regulatory proteins in a regulatory transcription complex as illustrated in FIG. 2. The term “transcription factor” as used herein therefore means a regulatory protein that binds directly or indirectly to a gene regulatory sequence in the genome to regulate the transcription of a gene including, without limitation, general transcription factors and specific transcription factors associated with the regulation of particular gene(s) as well as enhancer, co-enhancer, repressor, co-repressor, mediator, DNA bending protein, chromatin remodeling proteins, DNA damage repair proteins, RNA polymerase proteins or other transcription regulatory proteins. Similarly, the term “transcription factor binding site” (TFBS) as used herein means a DNA binding site of a regulatory protein associated with transcription regulation of a gene including without limitation distal or proximal enhancer and repressor sequences as shown in FIG. 2.

TFBS sequences are typically less than 10 bp in length and cfDNA fragments of 35-80 bp will therefore cover TFBS flanking sequences. The term “flanking sequence” as used herein means a DNA sequence present in the genome and located near to a TFBS. For example, a DNA sequence within 20 or 50 or 100 or 200 bp upstream or downstream of a TFBS. It will be clear to those skilled in the art that flanking sequences of a particular TFBS in the genome, for example located within a gene promoter sequence, may include the binding sites of other regulatory proteins.

Suitable TFBS sequences may be determined experimentally, for example using classical Nuclease Accessible Site mapping methods to determine the DNA sequence(s) associated with transcription factors of interest in the tissue(s) of interest. In a typical experiment, chromatin is extracted from the cells of interest (for example a cancer cell, a healthy cell of the same tissue, and a haemopoietic cell) and digested using a suitable nuclease. The chromatin fragments produced by digestion are exposed to an antibody that binds specifically to the transcription factor of interest and the antibody bound DNA fragments are isolated and sequenced to identify the TFBS sequence(s) (optionally including flanking sequences) bound by the transcription factor. Classical nuclease accessibility methods have recently been improved upon and the art now includes methods, including CUT&RUN and other methods, which are simpler to perform and provide improved results (Skene and Henikoff, 2017). Any such methods will be suitable for use in the identification of suitable DNA sequences for use in the present invention.

Suitable transcription factors and TFBS sequences and flanking sequences for use with the method of the invention may also be selected using various genomic, transcription factor and cancer data bases, for example the ENSEMBL database which provides an annotated genome sequence for a number of species including humans, the Encyclopedia of DNA Elements or (ENCODE) database (https://www.encodeproject.org), the Transcription Factor (TRANSFAC) database (Matys et al, 2006), The Gene Transcription Regulation Database (GTRD) Version 18.01 (http://gtrd.biouml.org), the Human Transcription Factors database Version 1.01 (http://humantfs.ccbr.utoronto.ca), the NIH Genomics Data Commons database (https://gdc.cancer.gov), The Cancer Genome Atlas (TCGA) (https://www.cancer.gov/about-nci/organization/ccg/research/structural-genomics/tcga), the UCSC Xena Browser (https://atacseq.xenahubs.net) and the Human Protein Atlas database (https://www.proteinatlas.org) which provides data on the healthy tissues in which a transcription factor is expressed as well as its expression in cancer diseases, as well as other databases.

The use of these databases for the characterization of transcription factors and associated TFBS sequences and flanking sequences for use in methods of the invention, can be illustrated with reference to a few of these databases as an example. The TRANSFAC database provides data on many thousands of human and other eukaryotic transcription factors. Details provided for each transcription factor include the number of TFBSs it binds to in the genome, lists of genes whose transcription it regulates, the sequence and genomic position of TFBSs associated with each regulated gene, details of other transcription factors that operate with it in a cooperative manner to regulate transcription, consensus TFBS DNA sequences, DBD details and cancer association. The use of this data in the context of the present invention is exemplified below for the transcription factors CDX2 and c-JUN for illustrative purposes. The TRANSFAC database lists 48 human CDX2 TFBSs which regulate 26 specified genes. The CDX2 TFBS sequences are provided as well as their genomic location and the genes regulated by each. The flanking sequences for each CDX2 TFBS can be determined by reference to the ENSEMBL human genome database for the sequence at each genomic location. Consensus CDX2 TFBS sequences are also provided. Similarly, The TRANSFAC database lists 265 human c-JUN TFBSs which regulate 166 specified genes. The c-JUN TFBS sequences are provided as well as their genomic location and the genes regulated by each. The flanking sequences for each c-JUN TFBS can be determined by reference to the ENSEMBL human genome database for the sequence at each genomic location. Consensus c-JUN TFBS sequences are also provided.

CTCF (also called CCCTC-binding factor) is an evolutionarily conserved zinc finger transcription factor that binds through a combination of 11 zinc fingers to a large number of sites in the genome and has a critical role in genome function. An investigation of CTCF binding sites in the human genome identified 77,811 distinct binding sites across 19 different cell types (Wang et al, 2012). 27,662 of the 77,811 binding sites were found to be occupied in all 19 cell types investigated. CTCF binding of the remaining 50, 149 binding sites exhibited tissue specificity. The 19 cell types investigated included 12 normal cell types and 7 cancer or EBV-immortalised cell lines representing colorectal cancer (Caco-2), cervical cancer (HeLa-S3), hepatocellular cancer (HepG2), neuroblastoma (SK-N-SH_RA), retinoblastoma (WERI-RB-1) and EBV-transformed lymphoplastoid (GM06990). CTCF binding at 1,236 binding sites was found to be specific to cancer cell lines. Occupancy of 195 of these binding sites occurred in normal cell lines but not in immortalized cancer cells. Occupancy of 1041 of these binding sites occurred in immortalized cancer cell lines but not in normal cells including epithelia, fibroblasts and endothelia (Liu et al, 2017).

Therefore, a transcription factor and/or TFBS may be selected experimentally or from the literature and/or from databases, such as The Human Protein Atlas database, as useful in methods of the invention. The transcription factor may be characterized in terms of (i) the healthy and diseased tissues in which it is expressed, (ii) the genes regulated in those cells or tissues, (iii) the TFBS sequences to which it binds in those tissues and (iv) other factors with which it cooperates by co-binding on a TFBS for transcriptional regulation. This characterization may be used to identify the healthy or diseased tissue or cells of origin of chromatin fragments and/or cfDNA fragments in a body fluid sample, by the methods described herein.

Similarly, experimental data relating to chromatin fragments and/or cfDNA sequences in body fluid samples may be interpreted using these databases to identify all or part of a TFBS sequence, optionally including flanking sequences, included in a cfDNA fragment. This data may then be used to identify the tissue or cells of origin of the cfDNA fragment.

In addition, there are many publications on transcription factors and cancer in the literature that list transcription factors useful in methods of the invention. For example Lambert et al, 2018 lists 294 known oncogenic transcription factors and regulators. Gurel et al, 2010 describes the transcription factor NKX3.1 as a marker for prostate cancer, Darnell, 2002 lists a number of oncogenic transcription factors including STAT3, 5, STAT-STAT, GR, IRF, TCF/LEF, β-catenin, NF-κB, NOTCH (NICD), GLI, c-JUN, bZip proteins (including c-JUN, JUNB, JUND, c-FOS, FRA, the ATFs and the CREB-CREM family), the cEBP family, ETS proteins and the MAD-box family, Vaquerizas et al, 2009 describe a number of tissue specific transcription factors useful in methods of the invention. Ulz et al, 2019 describes transcription factors such as the epithelial transcription factor GRHL2 which is present in many cancer types but not in hematological tissues as well as AR (Androgen Receptor), NKX3-1 and HOXB13. Corces et al, 2018 describe a number of cancer specific and tissue specific transcription factors including NR5A1, TP63, GRHL1, FOXA1, GATA3, NFIC, CDX2, RFX2, ASCL1, PAX2, HNF1A, NKX2.A, PHOX2B, DRGX, HOXB13, AR, MITF, HNF4 and POU5F1. Said references are herein incorporated by reference.

Suitable TFBS sequences, optionally including flanking sequences, for use with the invention may also be determined experimentally. For example, the patterns of small (e.g. 35-80 bp) cfDNA fragments present in samples obtained from patients diagnosed with, or without, a known disease state may be determined experimentally. The data may be used to generate TFBS loci or patterns of TFBS loci that are selectively present in samples obtained from diseased patients. This will generate a cfDNA TFBS biomarker or biomarker panel characteristic of the disease.

It is well known that transcription factor expression is altered in disease. Thus, a method of the invention may relate to a transcription factor whose expression is upregulated in disease, and/or inappropriately expressed in a disease tissue, for example a cancer tissue, when usually not highly expressed in said (healthy) tissue.

The chromatin fragments present in the circulation of healthy subjects are predominantly of hematopoietic origin. Thus, a method of the invention also relates to the inappropriate presence of a circulating chromatin fragment comprising a transcription factor together with associated DNA which is not expressed, or expressed at a low level, in healthy haemopoietic tissues but is expressed in a diseased tissue or a non-hematopoietic tissue. The presence of a chromatin fragment containing a transcription factor together with associated DNA in a sample, may be inferred by the detection of a cfDNA sequence related to its TFBS, optionally including flanking DNA sequences, following removal of nucleosome bound cfDNA by a method of the invention.

For example, many cancer diseases are derived from epithelial tissues. The epithelial transcription factor GRHL2 is expressed in multiple epithelial tissues as well as in many epithelial tissue derived cancer diseases, but is not expressed in hematopoietic tissues. The presence of GRHL2 in the circulation indicates the presence of an epithelial derived cancer, for example a colorectal, prostate, lung or breast cancer. Thus, methods of the invention may be used to detect the presence of a cancer per se. This may be used in conjunction with analysis of other TFBS sequences, and optionally flanking sequences, for lineage specific transcription factors and/or lineage specific combinations of transcription factors in a body fluid sample, to identify the organ of origin of the cancer. Any transcription factor, through its binding site sequences in the genome, may therefore be useful in methods of the invention. Preferred embodiments utilize TFBS sequences, optionally including flanking sequences, associated with transcription factors that are present in chromatin fragments at elevated levels in a body fluid of diseased subjects (over levels found in other subjects) and are partially or wholly tissue and/or disease specific, and have multiple response elements in the genome.

Therefore, in one embodiment, the transcription factor employed is disease specific (i.e. the level of circulating cfDNA fragments including its TFBS sequences is elevated in disease). In one embodiment, the transcription factor is tissue specific. In one embodiment, the transcription factor binds at more than one position in the genome, such as more than 5, more than 10, more than 100 or more than 1000 positions in the genome.

Transcription factors may be classified by binding domain (e.g. see Vaquerizas et al, 2009 which is incorporated herein by reference). In one embodiment, the transcription factor comprises a DNA binding domain selected from: a homeodomain, a HLH, a bZip, a NHR, a Forkhead, a P53, a HMG, an ETS, alPT/TIG, a POU, a MAD, a SAND, a IRF, a TDP, a DM, a Heat shock, a STAT, a CP2, a RFX, an AP2 or a zinc finger (e.g. zinc finger C₂H₂ or zinc finger GATA) binding domain.

There are three main groups of transcription factors which are currently recognized as being particularly important in cancer. The first group is the nuclear hormone receptor group which includes the estrogen receptor, the androgen receptor, the progesterone receptor, the glucocorticoid receptor, the thyroid receptor and the retinoic acid receptor. The nuclear hormone receptor group of transcription factors are cell surface receptors which can be regarded as inactive or latent transcription factors that may be activated by ligand binding. For example, the estrogen receptor is activated by binding to estrogen. Ligand binding results in migration of the nuclear hormone receptor to the nucleus where it binds to the target DNA sequence (for example, the estrogen receptor binds to the estrogen response element) and up or down regulates genes associated with the DNA target sequence (for example, estrogen regulated genes).

The second group of transcription factors that are known to be important in the initiation and development of cancer are the signal transducers and activators of transcription (STATs). These are latent cytoplasmic transcription factors that may be activated by a large variety of molecular triggers in the cytoplasm and/or at the cell surface. STAT activation typically involves a cascade of biochemical events in the cytoplasm such as kinase reactions, proteolysis reactions and protein-protein interactions that result in entry to the nucleus of a protein, or protein complex, that modulates transcription of target genes. Often the biochemical cascade leading to activation of transcription, is triggered by receptor binding of a ligand at the cell surface including for example, binding of a cytokine moiety by a cytokine receptor, or binding of a growth factor such as epidermal growth factor or platelet derived growth factor, by a growth factor receptor, or by binding of a peptide or protein to a G protein-coupled receptor.

The third group of transcription factors important in cancer are resident nuclear proteins whose transcriptional effects are typically activated by a cascade of biochemical events involving serine kinase reactions. There are hundreds of serine kinase moieties and hundreds of nuclear proteins that are targets for serine kinases.

It will be clear to those skilled in the art that cfDNA fragments comprising (i.e. including or containing) a TFBS related to any transcription factor involved in the initiation, development or maintenance of cancer, such as transcription factors in the three groups described above, will be useful in the methods of the present invention. Some transcription factors, or transcription factor families, with known roles in cancer, or known to be elevated in cancer diseases include for example, without limitation, STAT, particularly STAT3, STAT5 and STAT-STAT dimer moieties, NF-κB, β-catenin, γ-catenin, Notch and notch intracellular domain (NICD), GLI, c-JUN, JUNB, JUND, c-FOS, FRA, ATF, CREB-CREM, cEBP, ETS, MYC, N-MYC, MAX, E2F, interferon regulatory factor (IRF), T-cell factors (TCF), lymphocyte enhancer factors (LEF), EN2, GATA3, CDX2, PAX8, WT1, NKX3.1, P63 (TP63) or P40 and helix-loop-helix proteins (Darnell, 2002). All such transcription factors may be useful in methods of the invention.

It has been found that many transcription factors are lineage specific and associated with specific tissues and/or cancers, for example; a transcription factor that is always or commonly expressed in certain tissues or cancers but rarely or never expressed in other tissues or cancers. Methods of the invention may be used to detect a TFBS sequence, optionally including flanking sequences, that may be used as a tissue specific and/or cancer specific biomarker.

Thyroid transcription factor 1 (TTF-1) is selectively expressed during embryogenesis in the thyroid, the diencephalon, and in respiratory epithelium. TTF-1 is expressed in tissue samples taken from neuroendocrine and non-neuroendocrine lung carcinomas but its frequency of expression varies markedly among different histologic subtypes. TFBS sequences found in ctDNA by methods of the invention may therefore also be used to identify cancer types.

PAX8 is a transcription factor involved in the embryogenesis of the thyroid gland, kidney, and mullerian system. PAX8 shows a high level of expression in tissue samples taken from nonmucinous ovarian carcinomas, serous, endometrioid, clear cell, and transitional cell carcinomas. PAX8 is also expressed in endometrioid adenocarcinomas, uterine serous carcinomas, endometrial clear cell carcinomas as well as in ductal and lobular breast carcinoma tissues.

CDX2 is a lineage specific transcription factor with a key role in controlling the proliferation and differentiation of intestinal epithelial cells and is expressed in almost all colorectal adenocarcinoma tissue samples.

NKX3.1 is required for normal prostate development and is a known marker expressed in almost all prostate cancers.

GATA3 is active in transcription as early as the fourth week of human gestation. GATA3 is highly expressed in tissue samples taken from breast carcinomas, particularly estrogen receptor positive breast cancer tissue samples, and urothelial carcinomas and transitional cell carcinomas.

WT1 plays an important role in embryo development. WT1 is a good marker of ovarian cancer tissue and is expressed by a limited range of healthy adult tissues.

EN2 has a role in embryological development and is expressed in a range of cancers but in few adult healthy tissues. The presence of EN2 in the urine has been used as the basis for a urine test for the detection of prostate cancer.

Other transcription factor binding sites may be useful in the methods of the invention. For example; Upstream Binding Factor (UBF) is a transcription factor that binds to the ribosomal RNA gene promoter and activates transcription mediated by RNA polymerase I. UBF expression is known to be elevated in the tissue of some cancers. Many other such examples undoubtedly exist and are suitable transcription factors for use with methods of the present invention. Moreover, RNA polymerase I and RNA polymerase III are also elevated in cancers. These moieties are responsible for the transcription of tRNA and ribosomal RNA genes to provide the cellular machinery required for elevated and rapid protein production, growth and cellular replication characteristic of cancer cells and tissue. In further embodiments of the invention a method is provided for the detection or measurement of DNA binding sequences related to UBF, RNA polymerase I or RNA polymerase III binding in cell free chromatin fragments in a body fluid sample.

In some embodiments, the presence of a protein transcription factor in a body fluid chromatin fragment is not specific to a particular tissue or disease because the transcription factor may be expressed in multiple cell and tissue types. Thus, methods of the invention are also able to detect TFBS associated with transcription factors that are commonly expressed, i.e. a transcription factor which is expressed in more than 5, more than 10, more than 15, more than 20 or more than 30 tissue types. Detection of TFBS sequences associated with such transcription factors are also useful in methods of the invention where a TFBS sequence occurs in different genomic locations, for example in different gene promoters, in different tissues or in different disease conditions. Therefore the TFBS sequence and TFBS flanking sequences confer tissue and/or disease specificity to methods of the invention. One advantage of this embodiment is that the number of such locations may be large. For example 1041 CTCF TFBS locations are specifically occupied in cancer diseases. Similarly, differential occupation of large numbers of locations occurs for other highly expressed transcription factors including, for example without limitation, c-myc, n-myc, ER, AR, PR and many others.

Transcription factors bind to their DNA target sequence in a highly cooperative fashion with many other factors including other transcription factors, cofactors, co-activators, co-repressors, RNA polymerase moieties, elongation factors, chromatin remodeling factors, mediators, STAT moieties, UBF and others. This means that circulating chromatin fragments may comprise a larger gene regulation complex including any or all of a nucleosome with associated DNA, a nuclear hormone receptor, a steroid or other hormone bound to a nuclear hormone receptor, other transcription factors, cofactors, co-activators, co-repressors, RNA polymerase moieties, elongation factors, chromatin remodeling factors, mediators, STAT moieties or cytokine factors or cytokine related factors bound to a STAT moiety, UBF or any other moieties associated with such a gene regulation or transcription complex.

In addition any non-histone protein which binds to DNA in chromatin will be suitable for use in methods of the invention, including chromatin remodeling proteins, genetic and epigenetic reading, writing and deleting proteins, proteins involved in RNA transcription (for example; RNA polymerase proteins), chromatin architectural proteins and structural chromatin proteins (for example DNA bending proteins).

The term “binding agent” refers to ligands or binders, such as naturally occurring, recombinant or chemically synthesized compounds, capable of specific binding to a nucleosome. A ligand or binder according to the invention may comprise a peptide, a protein, an antibody or a fragment thereof, or a synthetic ligand such as a plastic antibody, or an aptamer or oligonucleotide or a molecular imprinted surface or device, capable of specific binding to the nucleosome or other target. The antibody can be a monoclonal antibody or a fragment thereof capable of specific binding to the target. A ligand or binder according to the invention may be labelled with a detectable marker, such as a luminescent, fluorescent, enzyme or radioactive marker; alternatively or additionally a ligand according to the invention may be labelled with an affinity tag, e.g. a biotin, avidin, streptavidin or His (e.g. hexa-His) tag. In one embodiment, the binding agent is selected from: an antibody, an antibody fragment or an aptamer. In a further embodiment, the binding agent used is an antibody. The terms “antibody”, “binding agent” or “binder” are used interchangeably herein.

In one embodiment, the sample is a biological fluid (which is used interchangeably with the term “body fluid” herein). Any body fluid sample type may be used for the invention including without limitation; blood, plasma, menstrual blood, endometrial fluid, feces, urine, saliva, mucous, semen and breath, e.g. as condensed breath, or an extract or purification therefrom, or dilution thereof. Biological samples also include specimens from a live subject, or taken post-mortem. The samples can be prepared, for example where appropriate diluted or concentrated, and stored in the usual manner. In a preferred embodiment, the biological fluid sample is selected from: blood or serum or plasma. It will be clear to those skilled in the art that the detection of chromatin fragments in a body fluid has the advantage of being a minimally invasive method that does not require biopsy.

In one embodiment, the subject is a mammalian subject. In a further embodiment, the subject is selected from a human or animal (such as a companion animal or a mouse) subject. In a yet further embodiment, the subject is a human subject. In one embodiment the subject is pregnant. In one embodiment, the human subject is a non-embryonic subject (i.e. a human at any stage of development, other than an embryo). In a further embodiment, the human subject is an adult subject, i.e. greater than 16 years of age, such as greater than 18, 21 or 25 years of age. In an alternative embodiment, the subject is an animal subject. In a further embodiment, the animal subject is selected from a rodent (e.g. mouse, rat, hamster, gerbil or chipmunk), feline (i.e. a cat), canine (i.e. a dog), equine (i.e. a horse), porcine (i.e. a pig) or bovine (i.e. a cow) subject.

It will be understood that the uses and methods of the invention may be performed in vitro or ex vivo.

According to a further aspect of the invention there is provided a method for detecting or diagnosing a disease in an animal or a human subject which comprises the steps of:
(i) removing a nucleosome from a body fluid sample obtained from the subject;
(ii) detecting, analyzing or measuring DNA associated with a cell free chromatin fragment in the remaining sample; and
(iii) using the DNA level and/or DNA sequence detected in step (ii) to identify the disease status of the subject.

In one embodiment of the invention, the presence of a DNA fragment in a sample is used to determine the optimal treatment regime for a subject in need of such treatment.

According to a further aspect of the invention there is provided a method for assessment of an animal or a human subject for suitability for a medical treatment which comprises the steps of:
(i) removing a nucleosome from a body fluid sample obtained from the subject;
(ii) detecting, analyzing or measuring DNA associated with a cell free chromatin fragment in the remaining sample; and
(iii) using the DNA level and/or DNA sequence detected in step (ii) as a parameter for selection of a suitable treatment for the subject.

According to a further aspect of the invention there is provided a method for monitoring a treatment of an animal or a human subject which comprises the steps of:
(i) removing a nucleosome from a body fluid sample obtained from the subject;
(ii) detecting, analyzing or measuring DNA associated with a cell free chromatin fragment in the remaining sample;
(iii) repeating the detection, analysis or measurement of DNA associated with a cell free chromatin fragment in the remaining sample after removal of a nucleosome from a body fluid sample obtained from the subject on one or more occasions; and
(iv) using any changes in the DNA level and/or DNA sequence detected in step (iii) compared to step (ii) as a parameter for any changes in the condition of the subject.

A change in the level of the measured DNA level and/or DNA sequence associated with a cell free chromatin fragment containing a transcription factor detected in the test sample relative to the level or sequence detected in a previous test sample taken earlier from the same test subject may be indicative of a beneficial effect, e.g. stabilization or improvement, of said therapy on the disorder or suspected disorder. Furthermore, once treatment has been completed, the method of the invention may be periodically repeated in order to monitor for the recurrence of a disease.

In one embodiment, the treatment is for the treatment of cancer, an autoimmune disease or an inflammatory disease.

The cfDNA sequence associated with a TFBS or other regulatory binding site detected by methods of the invention, may be detected or measured as one of a panel of measurements. Therefore, in one embodiment, the DNA level and/or DNA sequence is detected or measured as one of a panel of measurements. For example, in combination with other DNA markers, or with any other biomarkers.

According to a further aspect of the invention there is provided a method for detecting or measuring a DNA sequence in a DNA fragment associated with a non-nucleosomal cell free chromatin fragment, either alone or as part of a panel of measurements, for the purposes of determining or assessing an animal or a human subject for suitability for a medical treatment, or for monitoring a treatment of an animal or a human subject, for example for use in subjects with an actual or suspected cancer or benign tumor.

The terms “detecting” and “diagnosing” as used herein encompass identification, confirmation, and/or characterization of a disease state. Methods of detecting, monitoring and of diagnosis according to the invention are useful to identify persons at high risk of disease (as, for example, hemoglobin in the stool is associated with an elevated risk of colorectal cancer), to confirm the existence of a disease, to monitor development of the disease by assessing onset and progression, or to assess amelioration or regression of the disease. Methods of detecting, monitoring and of diagnosis are also useful in methods for assessment of clinical screening, prognosis, choice of therapy, evaluation of therapeutic benefit, i.e. for drug screening and drug development.

Efficient diagnosis and monitoring methods provide very powerful “patient solutions” with the potential for improved prognosis, by establishing the correct diagnosis, allowing rapid identification of the most appropriate treatment (thus lessening unnecessary exposure to harmful drug side effects), and reducing relapse rates.

It will be understood that identifying and/or quantifying can be performed by any method suitable to identify the presence and/or amount of DNA, or a specific DNA sequence in a biological sample from a patient or a purification or extract of a biological sample or a dilution thereof. In methods of the invention, identifying and/or quantifying may be performed by sequencing or by measuring the concentration or frequency of a TFBS sequence in the sample or samples. Biological samples that may be tested in a method of the invention include those as defined hereinbefore. The samples can be prepared, for example where appropriate diluted or concentrated, and stored in the usual manner.

The TFBS specific DNA fragment may be directly detected. Alternatively, it may be detected directly or indirectly via interaction with a ligand or ligands such as a DNA molecule, a transcription factor or other ligand or a fragment thereof, capable of specifically binding the TFBS specific DNA fragment. Suitable ligands include DNA molecules of complementary sequence that may bind to the cfDNA by hybridization. The ligand or binder may possess a detectable label, such as a luminescent, fluorescent or radioactive label, and/or an affinity tag.

For example, detecting and/or quantifying can be performed by one or more method(s) selected from the group consisting of: PCR, DNA sequencing, gene chip hybridization analysis or by SELDI (-TOF), MALDI (-TOF), a 1-D gel-based analysis, a 2-D gel-based analysis, Mass spec (MS), reverse phase (RP) LC, size permeation (gel filtration), ion exchange, affinity, HPLC, UPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

It will be understood that detecting and/or measuring DNA may comprise, for example, hybridization or sequencing as described herein.

Use of an immunological method as described herein, including immunoprecipitation and removal of a nucleosome may involve any moiety that binds selectively to nucleosomes including an antibody, or a fragment thereof, or a nucleosome binding chromatin protein or peptide, or an engineered binder capable of specific binding to a nucleosome.

Use of a binder moiety that binds to nucleosomes containing linker DNA as described herein, may include any moiety that binds selectively to nucleosomes containing linker DNA including naturally derived proteins or peptides, expressed proteins, engineered proteins or re-engineered proteins. In addition, it may not be necessary to use the whole protein and truncated proteins or peptides may be used.

According to a further aspect of the invention, there is provided a biomarker identified by the method described herein.

Diagnostic or monitoring kits are provided herein for performing methods of the invention. Such kits will suitably comprise a nucleosome binder, and optionally reagents for DNA isolation, for DNA library preparation, for DNA amplification and optionally reagents for DNA sequencing or analysis and optionally a ligand for detection and/or quantification of the target cfDNA or biomarker, optionally together with instructions for use of the kit. Biomarker monitoring methods, biosensors and kits are also vital as patient monitoring tools, to enable the physician to determine whether relapse is due to worsening of the disorder. If pharmacological treatment is assessed to be inadequate, then therapy can be reinstated or increased; a change in therapy can be given if appropriate. As the biomarkers are sensitive to the state of the disorder, they provide an indication of the impact of drug therapy.

According to a further aspect of the invention there is provided a kit for the detection of a cfDNA fragment sequence comprising a nucleosome binder and reagents for the amplification and/or sequencing of DNA associated with said cfDNA sequence, optionally together with instructions for use of the kit in accordance with the methods described herein.

A further aspect of the invention is a kit for detecting the presence of a disease state, comprising a biosensor capable of detecting and/or quantifying one or more of the biomarkers as defined herein.

According to a further aspect, there is provided the use of a kit as defined herein for the diagnosis of cancer. According to a further aspect, there is provided the use of a kit as defined herein for the diagnosis of an inflammatory disease. According to a further aspect, there is provided the use of a kit as defined herein for the diagnosis of a prenatal disease.

According to a further aspect, there is provided a method of treating a disease in a subject in need thereof, wherein said method comprises the following steps:
(a) contacting a body fluid sample obtained from the human or animal subject with a binding agent which binds specifically to a nucleosome;
(b) detecting or measuring a DNA fragment not bound to the binding agent in step (a);
(c) using the presence, sequence or amount of DNA fragment as an indicator of the presence of the disease in the subject; and
(d) administering a treatment if the subject is determined to have the disease in step (c).

In one embodiment, the disease is cancer, an autoimmune or inflammatory disease (for example as described hereinbefore). In a further embodiment, the disease is cancer.

In one embodiment, the treatment administered is selected from: surgery, radiotherapy, chemotherapy, immunotherapy, hormone therapy and biological therapy.

According to a further aspect of the invention, there is provided a method of treating cancer in a subject in need thereof, wherein said method comprises the following steps:
(a) detecting or diagnosing cancer in the subject according to the method described herein; followed by
(b) administering an anti-cancer therapy, surgery or medicament to said individual.

In one embodiment, the subject is a human or an animal subject.

We now illustrate the invention with the following examples.

### Example 1

We coated Dynabeads M280 Tosyl activated magnetic beads with an antibody directed to bind to a histone H3 epitope located at amino acid position 30-33. This antibody was selected from a number of antibodies tested as it was observed to bind to both nucleosomes containing full histone tails and to nucleosomes with clipped histone tails.

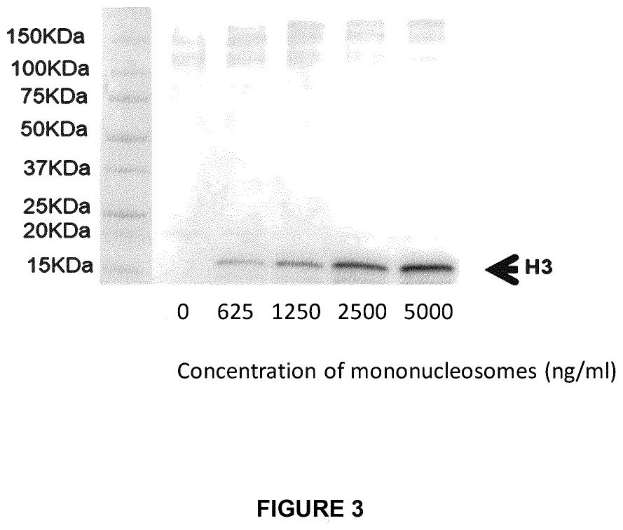

We added anti-H3 antibody coated magnetic beads (1 mg) to solutions containing a range of concentrations of recombinant mononucleosomes (0.5 ml). The beads were incubated with the nucleosomes at room temperature for 1 hour with gentle rolling of the tubes to maintain the beads in suspension. The beads were isolated magnetically and washed. Nucleosomes adsorbed to the beads were then removed by elution and analyzed by Western blot. The results demonstrate that the nucleosomes were adsorbed from solution by the magnetic beads in a dose dependent fashion as shown in FIG. 3.

### Example 2

Anti-H3 antibody coated magnetic beads were prepared and used as described in Example 1. We added anti-H3 antibody coated magnetic beads, as well as uncoated beads, to 8 human EDTA plasma samples as well as solutions containing a range of concentrations of recombinant mononucleosomes. The range of recombinant mononucleosomes concentrations was selected to include levels typically observed in human clinical samples.

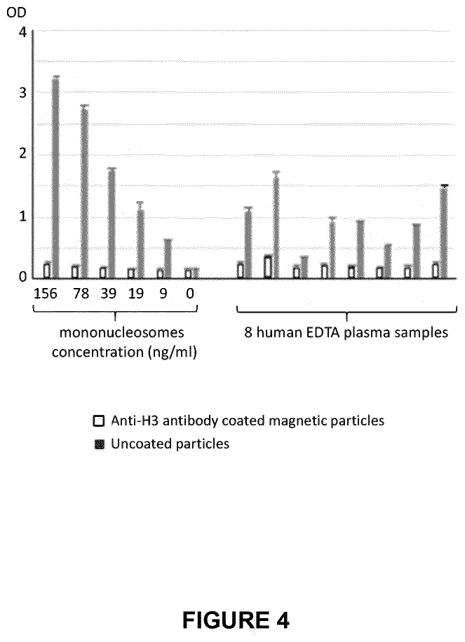

Preferred embodiments of the invention involve removal of all or most nucleosomes present in a sample prior to DNA analysis. Therefore, we tested for the presence of nucleosomes remaining in solution following incubation with magnetic beads using an ELISA for nucleosomes with an optical density (OD) readout. The results shown in FIG. 4, demonstrate that the level of recombinant mononucleosomes remaining in solution, following adsorption with anti-H3 antibody coated magnetic beads, was undetectable (had a similar OD to the control solution which contained no nucleosomes) whilst the levels in the solutions incubated with uncoated magnetic beads were unaffected leading to a normal ELISA dose response curve. Similarly, the level of nucleosomes remaining in solution in 8 human plasma samples tested, following adsorption with anti-H3 antibody coated magnetic beads, was also low or undetectable but was not affected by incubation with uncoated magnetic beads. These results demonstrate that nucleosomes may be quantitatively removed from human plasma samples using methods of the invention.

### Example 3

Plasma samples are taken from healthy subjects and from subjects with a variety of cancer diseases including, without limitation, cancer of the lung, colon, rectum, breast, prostate, liver, kidney, bladder, thyroid, head and neck, oral cavity, pharynges, esophagus, stomach, ovary, uterus, endometrium, skin and hematopoietic tissues (lymphomas and leukemias). The samples are depleted of nucleosomes as described in Example 2 and the remaining plasma sample is analyzed. DNA is isolated from the nucleosome depleted plasma samples, amplified to produce a library and sequenced. The DNA sequencing results are analyzed to identify transcription factor binding site (TFBS) sequences, plus flanking sequences, that are selectively present at elevated levels in the samples taken from cancer patients but absent from, or present at low levels in, the samples taken from healthy patients. Some of these DNA sequences are present in samples taken from multiple cancer disease types. Other DNA sequences are present in samples taken from patients with cancer of a particular organ or a particular type. The results are used to select transcription factors and TFBS sequences and flanking sequences for use with methods of the invention for use in relation to cancer per se or in relation to a particular cancer disease type.

### Example 4

The experiment described in Example 3 is repeated but the DNA sequencing results are analyzed for chromatin fragmentation patterns that characterize cancer or a particular cancer disease type.

### Example 5

Plasma samples are taken from healthy subjects and from subjects with prostate cancer. The samples are depleted of nucleosomes as described in Example 2. DNA is then isolated from the plasma samples, amplified and sequenced using a next generation sequencing instrument. The sequencing results are analyzed for the presence of the TFBS plus flanking sequences of the transcription factors NKX3.1 and GRHL2. Both the NKX3.1 and GRHL2 TFBS sequences are detected in the plasma samples taken from prostate cancer patients but they are not detected, or detected at a low level, in samples taken from healthy subjects.

### Example 6

The experiment described in Example 5 is repeated but the amplification of isolated DNA is performed using multiple sequence specific primers that are designed to amplify multiple promoter sequences that include the TFBS and flanking sequences of the transcription factors NKX3.1 and GRHL2. The results show that the quantity of DNA including at least one of the TFBS sequences amplified is high in samples taken from prostate cancer patients and low in samples taken from healthy subjects.

### Example 7

An experiment similar to that described in Example 6 is performed but using lung cancer samples and TFBS and flanking sequences associated with TTF-1 and GRHL2. The results show that the quantity of DNA including at least one of the TFBS sequences amplified is high in samples taken from lung cancer patients and low in samples taken from healthy subjects.

### Example 8

An experiment similar to that described in Example 6 is performed but using colorectal cancer samples and TFBS and flanking sequences associated with CDX-2 and GRHL2. The results show that the quantity of DNA including at least one of the TFBS sequences amplified is high in samples taken from colorectal cancer patients and low in samples taken from healthy subjects.

### Example 9

An experiment similar to that described in Example 6 is performed but using breast cancer samples and TFBS and flanking sequences associated with GATA3 and GRHL2. The results show that the quantity of DNA including at least one of the TFBS sequences amplified is high in samples taken from breast cancer patients and low in samples taken from healthy subjects.

### Example 10

The experiment described in Example 5 is repeated but the isolated DNA is contacted with magnetic solid phase immobilized transcription factor NKX3.1 and immobilized transcription factor GRHL2. The amount of DNA bound to the two magnetic transcription factors is measured by PCR. The results show that the quantity of DNA including at least one of the TFBS sequences amplified is high in samples taken from prostate cancer patients and low in samples taken from healthy subjects.

### Example 11

Plasma samples are taken from healthy subjects and from subjects with prostate, breast or lung cancer. The samples are depleted of nucleosomes as described in Example 2. DNA is then isolated from the plasma samples, amplified and contacted with a multiplicity of transcription factors immobilized on Luminex beads. The transcription factors NKX3.1, GATA3, TTF-1, CDX-2 and GRHL2 are each immobilized on beads of a different colour according the manufacturer's protocol. The amount of DNA bound to each transcription factor is measured by using a labelled anti-DNA antibody. The results show that the quantity of DNA bound to beads coated with NKX3.1 and GRHL2 is elevated in samples taken from prostate cancer patients whilst the binding to other beads is low, the quantity of DNA bound to beads coated with GATA3 and GRHL2 is elevated in samples taken from breast cancer patients whilst the binding to other beads is low, the quantity of DNA bound to beads coated with TTF-1 and GRHL2 is elevated in samples taken from lung cancer patients whilst the binding to other beads is low. In contrast the binding to all beads is low in samples taken from healthy subjects.

### Example 12

The experiment described in Example 11 is repeated with similar results, but immobilized NKX3.1, GATA3, TTF-1, CDX-2 and GRHL2 bound DNA is measured by PCR.

### Example 13

We coated a monoclonal antibody directed to bind histone H3 onto magnetic beads (MyOne TosylActivated Dynabeads™) using standard methods. Briefly, the monoclonal antibody was incubated with magnetic beads (40 μg antibody/mg of bead) in 0.1M Borate Buffer pH9.5 containing 1M Ammonium Sulfate for 18 hours at 37° C. in a rolling bottle to maintain suspension of the beads. The beads were sedimented and the supernatant was decanted. The beads were resuspended and incubated for 1 hour at 37° C. in a blocking buffer of phosphate buffered saline pH7.4 (PBS) containing 0.1% Tween 20 and 1% bovine serum albumin (BSA). The beads were then sedimented, washed twice with PBS containing 0.1% Tween 20 and 1% BSA and stored in PBS containing 0.1% Tween 20, 1% BSA and a preservative.

An EDTA plasma sample collected from a patient diagnosed with CRC (2.5 mL) was incubated with magnetic beads (0.15 mL, 10 mg/ml) for 1 hour at room temperature in a tube with rolling to maintain suspension of the particles. The magnetic particles were sedimented and removed. The remaining nucleosome depleted sample was retained.

The DNA in the nucleosome depleted sample, as well as in the original untreated plasma sample, was then extracted using a commercially available DNA extraction kit (Qiagen QIAamp DSP circulating NA kit) according to the manufacturer's instructions.

The extracted cfDNA was amplified to produce a single strand library for sequencing using a commercially available kit (Claret Bio SRSLY NGS Library Prep Kit) according to the manufacturer's instructions.

The amplified cfDNA library was sequenced by Next Generation Illumina NovaSeq sequencing.

Sequenced reads, each representing a cfDNA fragment, were aligned to the human reference genome GRCh38/hg38 using the Illumina DRAGEN Bioinformatics pipeline (https://emea.illumina.com/products/by-type/informatics-products/dragen-bio-it-platform.html). The resulting alignment BAM files were used to create subsets of different fragment sizes (35-80 bp, 135-155 bp and 156-180 bp) using Sequence Alignment/Map SAMtools (Li et al, 2009). Read coverage (the number of fragments found to cover a specific gene locus) was calculated using a bin size of 1 bp (the highest resolution possible). Read coverage was normalized to the total number of reads mapped to the human genome with the RPGC (reads per genome coverage) using the deepTools bamCoverage.

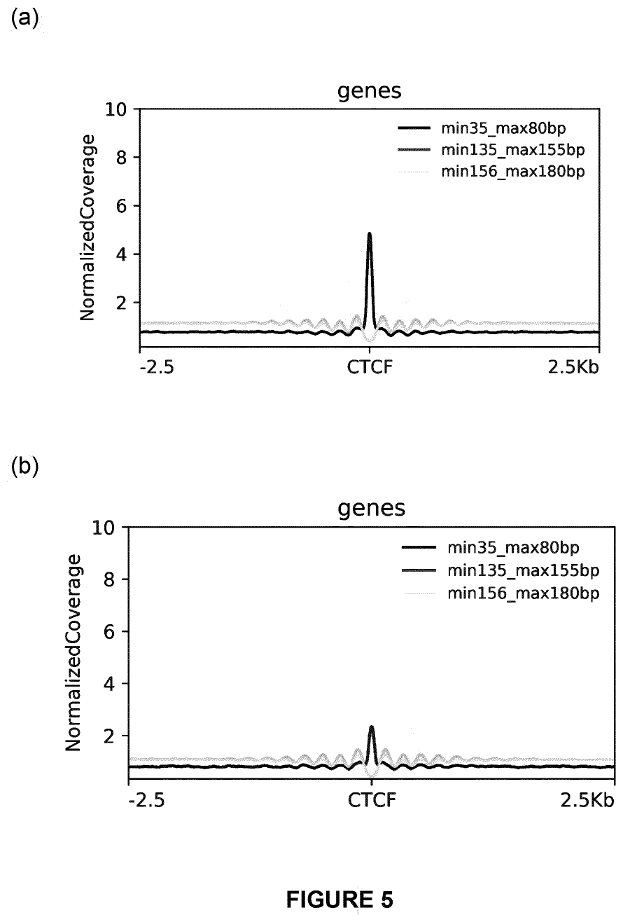

CTCF is often used as a model transcription factor because it is well characterized with 9780 known and published CTCF TFBS sequences (Kelly et al, 2012). Results for the coverage at the loci of 9780 published CTCF binding sites by short 35-80 bp cfDNA fragments, consistent with sizes expected for DNA fragments associated with CTCF, in comparison to coverage by longer cfDNA fragments, consistent with sizes expected for circulating mononucleosome association (135-155 bp and 156-180 bp), is shown in FIG. 5(a). The coverage is shown over a 5000 bp range including 2500 bases upstream and downstream of the CTCF binding site location. We observed a strong peak of coverage by small 35-80 bp cfDNA fragment binding at exactly the genomic positions of the CTCF TFBS loci reported by Kelly et al, 2012. Because the sequenced library was produced from cfDNA after removal of nucleosomes, the cfDNA library contained few nucleosomes and the nucleosome positioning signal was low. The amplitude of the 35-80 bp cfDNA fragment coverage peak at the CTCF TFBS loci in the genome (approximately 5 in FIG. 5(a)) is much larger than the amplitude of the periodic nucleosome positioning peaks (approximately 0.25). This low background feature produces an enhanced 35-80 bp signal.

By contrast, the cfDNA library obtained for the same sample with no treatment to remove nucleosomes showed a smaller amplitude peak for 35-80 bp cfDNA fragment coverage peak at the CTCF TFBS loci in the genome (approximately 0.7 in FIG. 5(b)) with a similar amplitude of the periodic nucleosome positioning peaks (approximately 0.25). This demonstrates that methods of the invention successfully remove nucleosome associated background cfDNA signals from liquid biopsy methods providing improved sensitivity for fragmentomics cfDNA analysis methods and other cfDNA analysis methods.

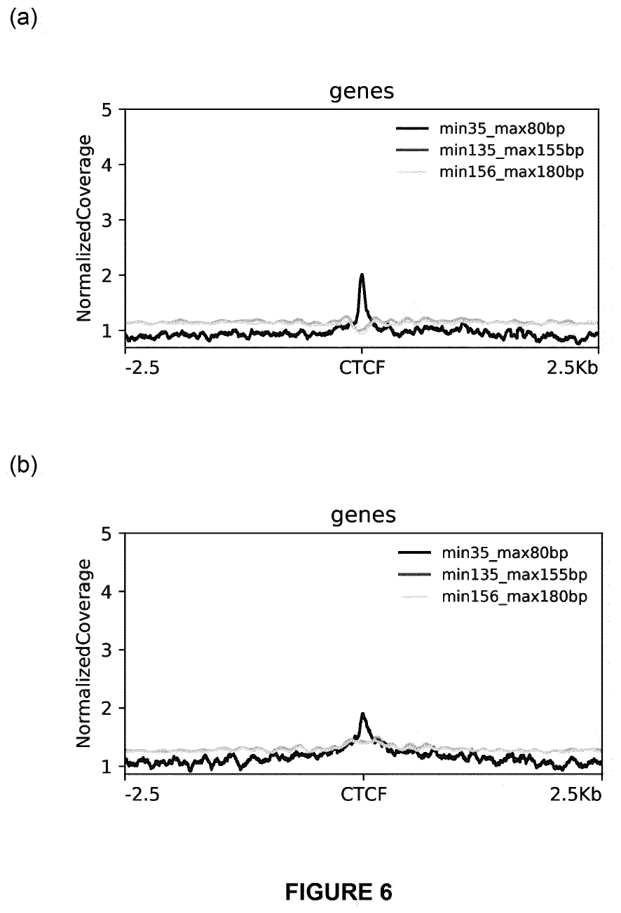

We then repeated the analysis for 1041 CTCF TFBS known to be occupied selectively in immortalized cancer cells (Liu et al, 2017) and not in healthy cells. The results shown in FIG. 6(a) show that there was a clear fragment coverage peak for 35-80 bp cfDNA fragment binding to the 1041 cancer specific CTCF TFBS sequences with a low background nucleosome periodicity signal. This indicates CTCF occupancy of the cancer specific TFBS loci and hence also indicates a tumor cell origin for those cfDNA fragments. Again, the cfDNA library obtained for the same sample with no treatment to remove nucleosomes showed a less clear and smaller amplitude peak for 35-80 bp cfDNA fragment coverage peak at the 1041 CTCF TFBS loci (FIG. 6(b)).

The demonstration that CTCF associated cfDNA fragments were bound to cancer specific TFBS loci in a body fluid by ChIP-Seq is indicative of the presence of a cancer disease in the subject investigated and can be used as a biomarker in this manner. We conclude that the methods of the invention are successful for the identification of disease associated TFBS in plasma as a biomarker for disease.

### REFERENCES

Active Motif, Nat. Methods 3: 658 (2006), doi: 10.1038/NMETH907
Bohinski et al. Molecular and Cellular Biology, 14(9): 5671 (1994)
Corces et al. Science, 362(6413): eaav1898 (2018), doi: 10.1126/science.aav1898.
Crowley et al. Nat. Rev. Clin. Oncol. 10: 472-484 (2013), doi: 10.1038/nrclinonc.2013.110
Darnell, Nat. Rev. Cancer 2: 740-749 (2002), doi:10.1038/nrc906
Deligezer et al. Clinical Chemistry 54:7 1125-1131 (2008)
Dunbar, Clinica Chimica Acta 363 (1-2): 71-82 (2006), doi.org/10.1016/j.cccn.2005.06.023
Gurel et al. Am J Surg Pathol, 34(8): 1097-105 (2010), doi: 10.1097/PAS.0b013e3181e6cbf3.
Heinz et al. Mol. Cell 38(4): 576-89 (2010), doi: 10.1016/j.molcel.2010.05.004.
Holdenrieder & Stieber, Crit. Rev. Clin. Lab. Sci. 46(1): 1-24 (2009), doi: 10.1080/10408360802485875
Hu et al. J. Trans. Med. 17: 124 (2019), doi: 10.1186/s12967-019-1871-x
Jung et al. Clin. Chim. Acta 411(21-22): 1611-24 (2010), doi:10.1016/j.cca.2010.07.032
Kelly et al. Genome Res. 22: 2497-2506 (2012), doi: 10.1101/gr.143008.112.
Klenova et al. Nucleic Acids Res. 25(3): 466-473 (1997), doi.org/10.1093/nar/25.3.466
Lambert et al. Cell 172(4):650-665 (2018), doi:10.1016/j.cell.2018.01.029
Latil et al. Cell Stem Cell 20(2): 191-204.e5 (2017), doi:10.1016/j.stem.2016.10.018.
Lee et al. J. Mol. Med. (Berl). 85(12):1393-404 (2007), doi: 10.1007/s00109-007-0237-7
Li et al. Bioinformatics 25(16): 2078-2079 (2009), doi: 10.1093/bioinformatics/btp352
Lin et al. PLOS Genet. 3(6):e87 (2007), doi: 10.1371/journal.pgen.0030087.eor
Liu et al. Oncotarget 8(69): 114183-114194 (2017), doi: 10.18632/oncotarget.23172
Liu et al. EBioMedicine 41: 345-356 (2019), doi:10.1016/j.ebiom.2019.02.010
Maenhaut et al. 2015 In: Feingold, Anawalt, Boyce, et al., editors. Endotext. https://www.ncbi.nlm.nih.gov/books/NBK285554/
Mann et al. Curr. Top Dev. Biol. 88: 63-101 (2009), doi: 10.1016/S0070-2153(09)88003-4.
Mansson et al. Mol. Oncol. 15(11): 2868-2876 (2021), doi: 10.1002/1878-0261.13093
Matys et al. Nucleic Acids Res. 34: D108-D110 (2006), doi: 10.1093/nar/gkj143
Merabet and Mann, Trends Genet. 32(6): 334-347 (2016), doi: 10.1016/j.tig.2016.03.004.
Newman et al. Nat. Med. 20(5): 548-54 (2014), doi: 10.1038/nm.3519
Park et al. Oncol. Lett. 3(4): 921-926 (2012), doi: 10.3892/ol.2012.592
Pomerantz et al. Nat. Genet. 47(11): 1346-51 (2015), doi:10.1038/ng.3419.
Poorey et al. Science 342(6156): 369-72 (2013), doi: 10.1126/science. 1242369.
Ramírez et al. Nucleic Acids Res. 44(W1): W160-5 (2016), doi: 10.1093/nar/gkw257
Ralston, Do transcription factors actually bind DNA? DNA footprinting and gel shift assays. Nature Education 1(1): 121 (2008)
Sadeh et al. Nat. Biotechnol. 39: 586-598 (2021), doi.org/10.1038/s41587-020-00775-6
Sanchez et al. NPJ Genom. Med. 3: 31 (2018), doi:10.1038/s41525-018-0069-0
Skene and Henikoff, eLife 6:e21856 (2017), doi: 10.7554/eLife.21856.002
Snyder et al. Cell 164(1-2): 57-68 (2016), doi:10.1016/j.cell.2015.11.050
Ulz et al. Nat. Commun. 10(1): 4666 (2019), doi:10.1038/s41467-019-12714-4
Vad-Nielsen et al. Lung Cancer 147: P244-251 (2020), doi.org/10.1016/j.lungcan.2020.07.023
Vaquerizas et al. Nat. Rev. Genet. 10(4): 252-63 (2009), doi:10.1038/nrg2538
Wang et al. Genome Res. 22(9): 1680-8 (2012), doi: 10.1101/gr.136101.111
Zhang et al. Genome Biol. 9(9): R137 (2008), doi: 10.1186/gb-2008-9-9-r137
Zhou et al. BMC Genomics 18(1):724 (2017), doi: 10.1186/s12864-017-4115-6

1. A method of detecting a cell free DNA chromatin fragment including all or a part of a transcription factor binding site sequence in a body fluid sample obtained from a human or animal subject, comprising:
(i) contacting the body fluid sample with a binding agent which binds to a nucleosome; and
(ii) analyzing the DNA from the body fluid sample not bound to the binding agent in step (i).

2. A method of detecting a cell free DNA chromatin fragmentation pattern in a body fluid sample obtained from a human or animal subject, comprising:
(i) contacting the body fluid sample with a binding agent which binds to a nucleosome; and
(ii) analyzing the DNA from the body fluid sample not bound to the binding agent in step (i).

3. The method of claim 1, wherein the binding agent binds to a nucleosome core epitope.

4. The method of claim 1, wherein the binding agent binds to a nucleosome containing linker DNA.

5. The method of claim 4, wherein the binding agent is all or a part of histone H1 moiety or a chromatin binding protein.

6. The method of claim 4, wherein the binding agent binds to histone H1 or a component thereof.

7. The method of claim 1, wherein the binding agent is attached to a solid support.

8. The method of claim 2, wherein the DNA is analyzed for the presence of a transcription factor binding site and/or flanking sequence.

9. The method of claim 1, wherein the DNA is analyzed by PCR.

10. The method of claim 1, further comprising determining a presence, amount, sequence or fragmentation pattern of the DNA as an indicator of the disease state of the subject.

11.-23. (canceled)

24. A kit for the detection of a cfDNA fragment sequence, comprising: a nucleosome binder and reagents for one or more of amplification, sequencing or obtaining a fragmentation pattern of DNA associated with said cfDNA sequence, optionally together with instructions for use of the kit in the method according to claim 1.

25. A method of treating a disease in a subject in need thereof, wherein said method comprises the following steps:
(i) contacting a body fluid sample obtained from the human or animal subject with a binding agent which binds to a nucleosome;
(ii) detecting or measuring a DNA fragment not bound to the binding agent in step (i);
(iii) using the presence, sequence, amount or fragmentation pattern of the DNA fragment as an indicator of the presence of the disease in the subject; and
(iv) administering a treatment if the subject is determined to have the disease in step (iii).

26. The method according to claim 25, wherein the treatment administered is selected from: surgery, radiotherapy, chemotherapy, immunotherapy, hormone therapy and biological therapy.

27. (canceled)

28. The method of claim 2, wherein said contacting comprises contacting the body fluid sample with a binding agent which binds to a nucleosome containing linker DNA.

29. The method of claim 3, wherein the binding agent binds to histone H3.
